(19) United States
(12) Patent Application Publication
(10) Pub. No.: US 2009/0306400 A1
(43) Pub. Date: Dec. 10, 2009

(54) CONVERGENT PROCESS FOR THE SYNTHESIS OF TAXANE DERIVATIVES.

(76) Inventors: John T. Henri, Longmont, CO (US); James D. McChesney, Boulder, CO (US); Sylesh K. Venkataraman, Boulder, CO (US); Rodger L. Lamb, Westminster, CO (US); Jonathan E. Foster, Arvada, CO (US); Christian M. Sumner, Boulder, CO (US); Shangping Ye, Centennial, CO (US)

Correspondence Address: MORGAN, LEWIS & BOCKIUS LLP, 1701 MARKET STREET, PHILADELPHIA, PA, 19103-2921, US

(21) Appl. No.: 12/225,636

(22) PCT Filed: Mar. 26, 2007
(86) PCT No.: PCT/US2007/007687

§ 371 Date: Jul. 24, 2009

Related U.S. Application Data
(60) Provisional application No. 60/786,629, filed on Mar. 27, 2006.

(51) Int. Cl. C07D 233/02; C07D 305/14; C07D 309/02; C07F 9/06

(52) U.S. Cl. 548/334.5; 549/510; 549/358; 564/12

(57) ABSTRACT

The present invention is broadly directed to novel compounds useful for the synthesis of biologically active compounds, including taxane derivatives, and convergent processes for the preparation of these taxane derivatives and their intermediates.

### FIELD OF THE INVENTION

The present invention is broadly directed to novel compounds useful for the synthesis of biologically active compounds, including taxane derivatives, and convergent processes for the preparation of these taxane derivatives and their intermediates.

### BACKGROUND OF THE INVENTION

Various taxane compounds are known to exhibit anti-tumor activity. As a result of this activity, taxanes have received increasing attention in the scientific and medical community, and are considered to be an exceptionally promising family of cancer chemotherapeutic agents. For example, taxanes such as paclitaxel and docetaxel have been approved for the chemotherapeutic treatment of several different varieties of tumors. Paclitaxel is a naturally occurring taxane diterpenoid having the formula and numbering system for the taxane backbone as follows:

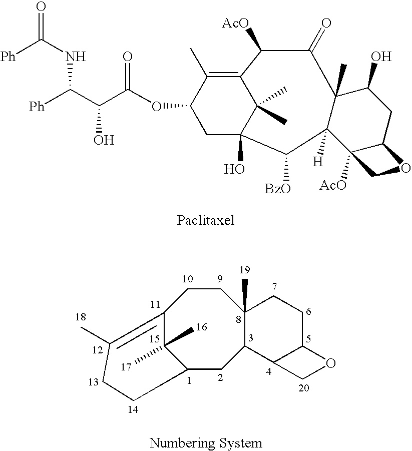

Since paclitaxel appears promising as a chemotherapeutic agent, chemists have spent substantial time and resources in attempting to synthesize paclitaxel and other potent taxane analogs. The straightforward implementation of the partial synthesis of paclitaxel or other taxanes, requires convenient access to chiral, non-racemic side chains and derivatives, an abundant natural source of baccatin III or closely related diterpenoid substances, and an effective means of joining the two units. Perhaps the most direct synthesis of paclitaxel is the condensation of Baccatin III and 10-deacetylbaccatin III of the formula:

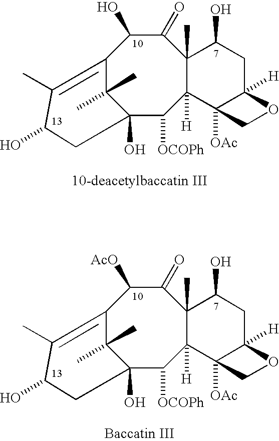

with the side chain:

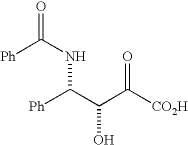

However, the esterification or coupling of these two units is difficult because of the C-13 hydroxyl group of both baccatin III and 10-deacetylbaccatin III are located within the sterically encumbered concave region of the hemispherical taxane skeleton.

Alternative methods of coupling the side chain to a taxane backbone to ultimately produce paclitaxel have been disclosed in various patents. For example, U.S. Pat. No. 4,929,011 issued May 8, 1990 to Denis et al. entitled “Process for Preparing Taxol”, describes the semi-synthesis of paclitaxel from the condensation of a (2R,3S) side chain acid of the general formula:

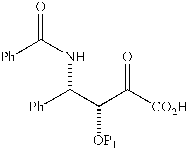

wherein P₁ is a hydroxyl protecting group with a taxane derivative of the general formula:

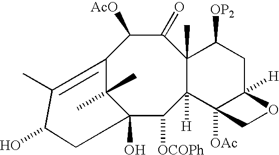

wherein P₂ is a hydroxyl protecting group. The condensation product is subsequently processed to remove the P₁ and P₂ protecting groups. In Denis et al., the paclitaxel C-13 side chain, (2R,3S) 3-phenylisoserine derivative is protected with P₁ for coupling with a protected baccatin III. The P₂ protecting group on the baccatin III backbone is, for example, a trimethylsilyl or a trialkylsilyl radical.

An alternative semi-synthesis of paclitaxel is described in U.S. Pat. No. 5,770,745 to Swindell et al. Swindell et al. disclose semi-synthesis of paclitaxel from a baccatin III backbone by the condensation with a side chain having the general formula:

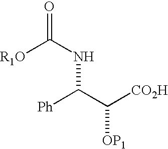

wherein R₁ is alkyl, olefinic or aromatic or PhCH₂ and P₁ is a hydroxyl protecting group.

Another method for the semi-synthesis of paclitaxel is found in U.S. Pat. No. 5,750,737 to Sisti et al. In this patent, C7-CBZ baccatin III of the formula

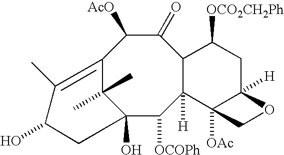

is esterified with a C3-N—CBZ-C2-O-protected (2R,3S)-3-phenylisoserine side chain of the formula:

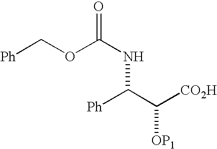

followed by deprotection, and C3′N benzoylation to produce paclitaxel.

Another taxane compound that has been found to exhibit anti-tumor activity is the compound known as docetaxel. This compound is also sold under the trademark TAXOTERE®, the registration of which is owned by Sanofi Aventis. Docetaxel has the following structure:

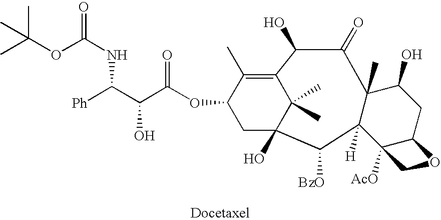

As noted in the above structure, docetaxel is similar to paclitaxel except for the t-butoxycarbonyl (t-Boc) group at the C3′ nitrogen position of the phenylisoserine side chain and a free hydroxyl group at the C10 position. Similar to paclitaxel, the synthesis of docetaxel is difficult due to the hindered C13 hydroxyl in the baccatin III backbone, which is located within the concave region of the hemispherical taxane skeleton. Several syntheses of docetaxel and related compounds have been reported in the Journal of Organic Chemistry: 1986, 51, 46; 1990, 55, 1957; 1991, 56, 1681; 1991, 56, 6939; 1992, 57, 4320; 1992, 57, 6387; and 993, 58, 255; also, U.S. Pat. No. 5,015,744 issued May 14, 1991 to Holton describes such a synthesis. Additional techniques for the synthesis of docetaxel are discussed, for example, in U.S. Pat. No. 5,688,977 to Sisti et al. and U.S. Pat. No. 6,107,497 to Sisti et al.

Due to the promising anti-tumor activity exhibited by both paclitaxel and docetaxel, further investigations have indicated that analogs and derivates within the taxane family may lead to new and better drugs having improved properties such as increased biological activity, effectiveness against cancer cells that have developed multi-drug resistance (MDR), fewer or less serious side effects, improved solubility characteristics, better therapeutic profile and the like.

While the existing procedures for synthesizing paclitaxel and docetaxel have merit, there is still a need for improved chemical processes for preparing these anti-cancer compounds and their derivatives in good yields. The present invention is directed to meeting these needs.

### SUMMARY OF THE EXEMPLARY EMBODIMENTS

According to the present invention, methods are described for use in producing taxanes, taxane analogs, and derivatives thereof. In one aspect, there is provided herein a new and efficient convergent synthesis for the preparation of compound 10 that provides the desired product in high overall yields, requiring a lower number of chemical and mechanical processing steps and provides the desired product in higher chemical purity.

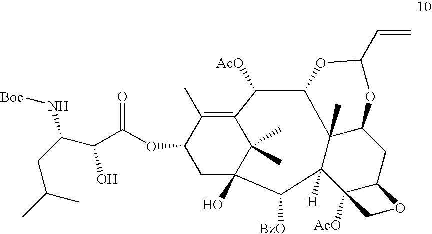

In one embodiment, there is provided a process for preparing compound 2 by the selective oxidation of a compound of formula 1. In particular, there is provided a process for preparing a 9,10-di-keto baccatin derivative 2:

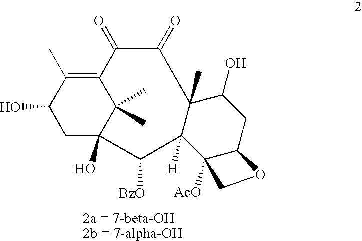

the process comprising contacting a 9-keto alcohol 1

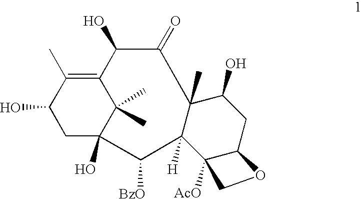

in the presence of CuCl₂ and a base. In one variation of the above process, the base is an amine base. In another variation, the amine base is TEA, and the process is carried out in an organic solvent or a mixture of solvents. In another variation of the above process, the mixture of solvents comprises EtOH and EtOAc, and the process is carried out below room temperatures to form the desired product 2 in less than about 5 hours, less than 3 hours or 1 hour or less. In another variation of the process, the reaction may be carried out in MeOH, IPAC, THE, EtOAc and mixtures thereof. In a particular variation of any one of the above, the process affords a mixture of 2a:2b in ratio of at least 95:5 and at least 85% yield. In a particular variation, the mixture of 2a:2b is optionally further purified by digestion of the crude reaction mixture to afford only La in at least 80% yield.

In one aspect as shown in FIG. 2, the 9-keto alcohol 1 is selectively oxidized to form the 9,10-di-ketone 2. The di-ketone 2 may be obtained as a mixture of the di-keto, 2a and 2b. In one variation of the process, the two isomers, 2a and 2b, may be separated to afford 2a, or the mixture of the isomers may be used as is in the subsequent step without separation. The mixture may be derivatized to form the corresponding protected alcohol, and a number of applicable alcohol protecting groups are disclosed, for example, in T. W. Greene and P. G. M. Wuts, Protective Groups in Organic Synthesis, 4^{th} Edition, John Wiley & Sons, New York (2007).

In one particular method, the mixture is derivatized to form the corresponding protected silyl ether, such as the triethylsilyl ether, by treating the mixture with TES-OTf (trifluoromethanesulfonic acid triethylsilyl ester), pyridine and NMP to form the 7,13-di-silyl ether 3. If desired, before the silylation step, the undesired isomer 2b may be separated from the desired isomer 2a using various methods known in the art, including column chromatography and crystallization. Alternatively, where a mixture of the isomers 2a and 2b are used as the starting mixture, the epimeric isomers of the corresponding di-silyl ether 3 obtained may be separated using standard procedures known in the art. However, because the isomer 2b forms the di-TES ether at a slower rate than the isomer 2a, the reaction condition may be adjusted accordingly to favor the formation of the diether 3.

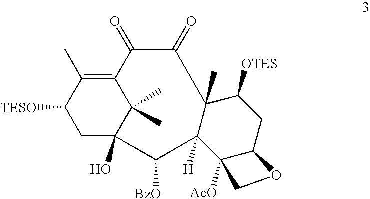

In another embodiment, there is provided a reaction for the preparation of a 9,10-di-ol baccatin derivative 4:

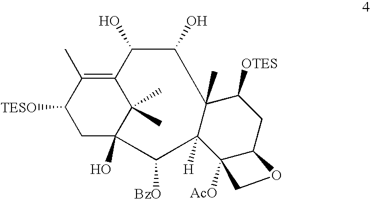

the process comprising contacting a 9,10-di-keto baccatin derivative 2, as a mixture of 2a and 2b, or 2a as a single isomer;

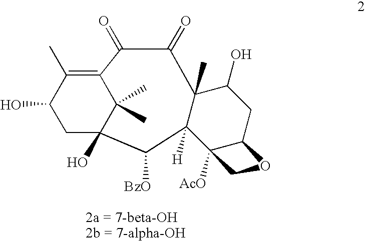

with a silylation reagent to form a di-silyl ether 3; and reducing the 9,10-di-ketone of the di-silyl ether 3 with a reducing reagent to form the 9,10-di-ol baccatin derivative 4. In one variation of the above process, the silylation reagent is TES-OTf, and NMP in pyridine and the di-silyl ether 3 is formed in at least 97% yield. In another variation of the above process, the silylation reagent is TES-OTf and pyridine in the presence or absence of a solvent. In another variation of the process, the reaction may be carried out in MeOH, IPAC, THF, EtOAc and mixtures thereof. In certain variations of the process, the silylation reagent used is TMS-OTf to form the corresponding di-TMS ether. In another variation of the above process, the reducing reagent is LiBH₄ and the reduction reaction is performed in THF/ethanol solvent to provide the 9,10-di-ol 4 in >90% yield. In a particular variation of the reduction reaction, the reducing reagent used is selected from the group consisting of NaBH₄, CaBH₄, LiAlH₄, K-SELECTRIDE and KS-SELECTRIDE in ether, such as THF. In the above process, the di-silyl ether 3 and the 9,10-di-ol 4 are both obtained as the di-silylated product with no detectable mono-silylated product.

The di-silyl ether 3 may be reduced to the corresponding 9,10-di-ol 4. Reduction may be performed using a hydride reducing agent, such as using NaBH₄ in an organic solvent. In one process of the above, reduction of the di-ketone may be accomplished using LiBH₄ in a solvent or solvent mixture, such as THF/EtOH to form the di-ol 4. The reaction may be performed at room temperature, or below room temperature, or at about 20° C. to about −10° C., more preferably at about 0° C. with other variations of solvent combinations such as DCM/EtOH etc. In another variation of the process, the reaction may be carried out in DCM, MeOH, IPAC, THF, EtOAc and mixtures thereof.

Using the methods described herein with the change in the particular substitutions on the baccatin derivative, the following compounds may be prepared:

| | |
|---|---|
| | A |
| 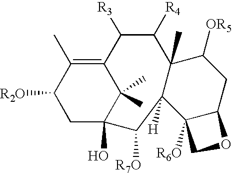 | |

| | R₂ | R₃ | R₄ | R₅ | R₆ | R₇ |
|---|---|---|---|---|---|---|
| A | H, TES, Bn, | α-OH, β- | α-OH, ═O, | H, TES, Bn, | CH₃CO, | CH₃CO, |
| | TMS, | OH, ═O, | CH₃CO, | TMS, | PhCO | PhCO |
| | iPrDMS, | CH₃CO, | PhCO, | iPrDMS, | CH₃OCO | |
| | TBDMS, | PhCO | α-OP_{12′}, | TBDMS, | | |
| | MDiPrS, | | β-OP₁₂. | MDiPrS, | | |
| | TBDPS, TPS | | | TBDPS, TPS, | | |
| | | | | R_{13′} | | |
| 2 | H | ═O | ═O | H | CH₃CO | PhCO |
| 2a | H | ═O | ═O | H (β-OH) | CH₃CO | PhCO |
| 2b | H | ═O | ═O | H (α-OH) | CH₃CO | PhCO |
| 3 | TES | ═O | ═O | TES | CH₃CO | PhCO |
| 4 | TES | α-OH | α-OH | TES | CH₃CO | PhCO |

“═O” also means oxo (or keto); and where R_{12′} and R_{13′} together with the oxygen atoms to which they are attached to form a cyclic C₁-C₆ alkyl acetal, a cyclic C₂-C₁₀ alkenyl acetal or a cyclic aryl acetal.

In another embodiment, there is provided a process for the preparation of an allylidene acetal 7 baccatin derivative 7:

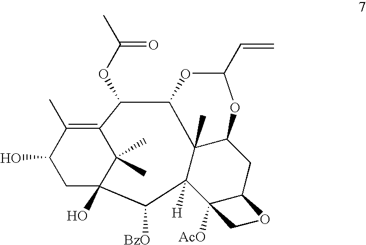

the process comprising contacting a di-ol 4 with an acylating reagent to form a 10-acylated alcohol 5;

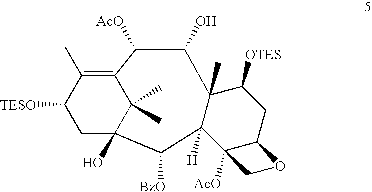

selective hydrolysis of the TES groups to form the corresponding tetra-ol 6; and

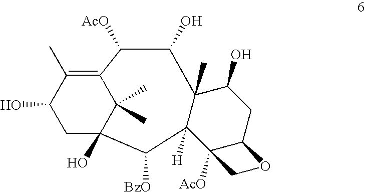

acetalization of the 7,9-di-ol of compound 6, to provide the allylidene acetal 7. In one variation of the above process, the acylation reagent is Ac₂O, TEA and DMAP in IPAC, and the selective hydrolysis is performed with acetic acid in aqueous methanol. In another variation of the process, the reaction may be carried out in IPAC, THF, EtOAc and mixtures thereof. In a particular variation of the process, the acylation reaction provides the 10-acylated alcohol 5 in >85% yield. In another variation of the process, the acetalization of the 7,9-di-ol of compound 6 is performed with acrolein diethyl acetal or acrolein dimethyl acetal in a polar or non-polar solvent to provide the allylidene acetal 7 and an acid selected from the group consisting of TFA, TFA/TFAA, CSA and CDSA. In a particular variation of the above process, the non-polar solvent is toluene, xylenes or DCM. In another aspect of the above process, the selective hydrolysis and the acetalization reaction steps are performed to form the allylidene acetal 7 without isolation of the intermediate compound 6.

In another embodiment, there is provided a variation of the above process for the preparation of an allylidene acetal baccatin derivative 7a or 7b:

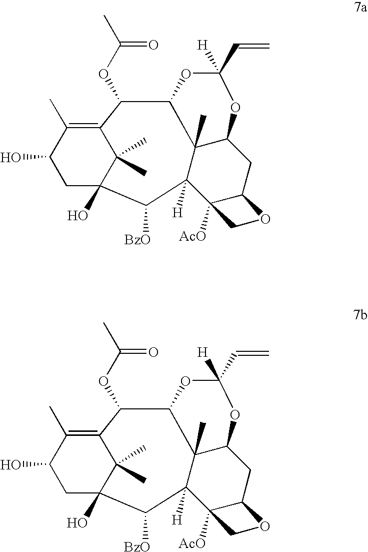

as substantially the pure diastereoisomer 7a or 7b, or as a mixture of the diastereoisomers 7a and 7b using the above method. The specific reaction conditions and reagents described above may be changed to favor one diastereoisomer over another, or the reaction conditions may be changed to afford a mixture of the diastereoisomers as desired. In one aspect of the above process, the process provide the substantially pure diastereoisomer 7a in >90% yield.

In another embodiment, there is provided compounds comprising the following formulae:

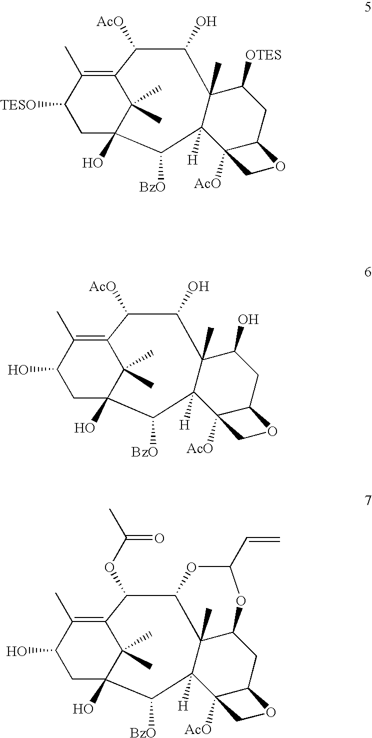

In FIG. 3, the di-ol 4 is converted to the corresponding 10-acylated alcohol 5 using an acylation agent such as acetic anhydride, TEA, DMAP and IPAC. Selective hydrolysis of the TES groups may be accomplished using, for example, AcOH in MeOH/H₂O, or in IPAc/MeOH, to afford the tetra-ol 6. Acetalization of the 7,9-di-ol of compound 6, preferably using acrolein diethyl acetal in an organic solvent, such as toluene, and TFA in an ice bath, provides the allylidene acetal 7 in good yields. Similarly, the acetalization may also be performed using acrolein dimethyl acetal in an organic solvent. In one variation of the process, the allylidene acetal 7 is prepared from the 10-acylated alcohol 5 without isolation of the intermediate tetra-ol 6.

In another variation of the process, the tetra-ol 6 is prepared by acetylation of the diol 4 and subsequent hydrolysis without isolation of the 10-acylated alcohol 5.

In yet another embodiment, there is provided a process for the preparation of compound 10:

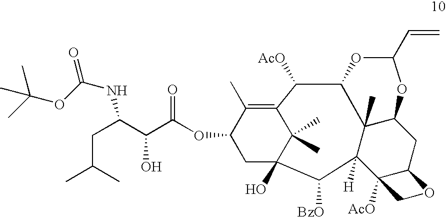

the process comprising contacting an allylidene acetal 7 with a side chain 8 under a coupling reaction condition to form a coupled intermediate compound 9;

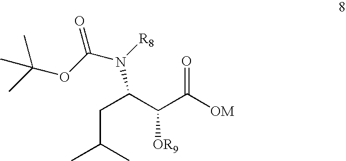

wherein R₈ and R₉ together with the nitrogen and oxygen to which they are attached form a cyclic 2,4-dimethoxy benzylidene N,O-acetal or a cyclic 2,6-dimethoxy benzylidene N,O-acetal, and M is H or an alkali metal selected from the group consisting of Li, Na and K;

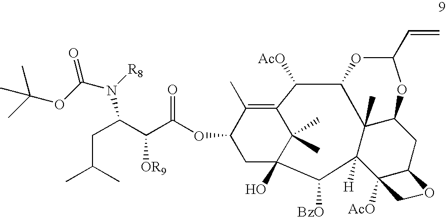

to form compound 9. Subsequent hydrolysis of compound 9 forms compound 10. In one variation of the process, the coupling reaction condition comprises contacting the allylidene acetal 7 with the side chain 8 in Piv-Cl, TEA, DMAP and THF or Piv-Cl, NMM, DMAP and THF for a sufficient amount of time to form compound 9 which is hydrolyzed to form compound 10 in >90% yield. In addition to NMM and DMAP, other amine bases may be employed, including DABCO, pyridine, DBN, DBU, and the like. As illustrated in FIG. 3, coupling of the allylidene acetal 7 with the acid 8a affords the coupled product 9a. Deprotection affords compound 10 in good yields.

In another embodiment, there is provided a compound comprising the formula 9:

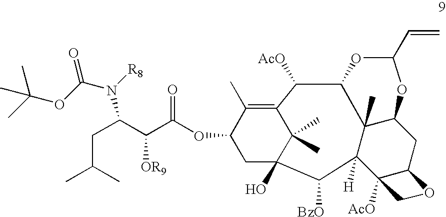

wherein R₈ and R₉ together with the nitrogen and oxygen to which they are attached form a cyclic 2,4-dimethoxy benzylidene N,O-acetal or a cyclic 2,6-dimethoxy benzylidene N,O-acetal. In one variation of compound 9, R₈ is hydrogen and R₉ is a hydroxyl protecting group, such as a silyl ether or a base labile ester such as an acetate, a phenoxy acetate and the like.

In another aspect of the process, the coupling reaction of the allylidene acetal 7 with the acid 8 affords the coupled product 9, which is not isolated, and the N,O-acetal is hydrolyzed in situ, as provided herein affords the product, compound 10 in good yields. The hydrolysis may be performed using an acid in an alcohol at low temperatures, such as hydrochloric acid in methanol at about −25° C. to 25° C., or at about −10° C. to −20° C., preferably about −15° C. This general procedure may be employed using either of the starting isomer 8a (2,4-dimethoxy isomer) or 8b (2,6-dimethoxy isomer), that forms the corresponding isomer 9a or 9b respectively. See FIG. 1.

As shown in FIG. 3, when the acid 8b is employed in the coupling reaction with compound 7, the resulting product 9b is formed as the coupled product. The N,O-acetal 8b may be prepared according to the procedure illustrated in FIG. 4 to provide the desired product in good yield. Similarly, the N,O-acetal isomer 8a may be prepared according to the procedure illustrated in FIG. 4 to provide the product in good yield. A method for preparing the intermediates, 12, 13 and 14, in FIG. 4 is disclosed in the Journal of Organic Chemistry, 2001, 66, 3330-3337, the reference of which is incorporated herein in its entirety.

According to the above, there is provided a process for the preparation of compound 10 comprising: a) selective oxidation of keto-alcohol 1 to afford compound 2a; b) protection of the 1,7,13-tri-hydroxy compound 2a to afford compound 3; c) selective reduction of compound 3 to provide di-ol 4; d) derivatizing di-ol 4 to form ester 5; e) deprotection of the protected ethers to form tetra-ol 6; f) acetalization of tetra-ol 6 to form acetal compound 7; g) coupling of compound 7 with compound 8a to afford compound 9a; and h) deprotection of compound 9a to form compound 10 as shown in the FIG. 1.

In another aspect of the above, there is provided a process for the preparation of compound 10, comprising: a) selective oxidation of keto-alcohol 1 to afford compound 2; b) protection of the 1,7,13-tri-hydroxy compound 2 to afford compound 3; c) selective reduction to provide di-ol 4; d) derivatizing di-ol 4 to form ester 5; e) deprotection of the silyl ethers to form tetra-ol 6; f) acetalization of tetra-ol 6 to form compound 7; g) coupling of compound 7 with compound 8a to afford compound 9a; and h) deprotection of compound 9a to form compound 10, as shown in FIGS. 2 and 3. In one variation of the process, the selective oxidation is performed with CuCl₂, TEA, EtOAc and EtOH. In a particular variation, the protection of 1,7,13-tri-hydroxy compound 2a is accomplished with TES-OTf, pyridine and NMP at −10 to 50° C. In another variation, the selective reduction of compound 3 is performed using LiBH₄ in THF/EtOH to form di-ol 4. In yet another variation of the above process, derivatizing 9,10-di-ol 4 to form ester 5 is performed using acetic anhydride, TEA, DMAP and IPAC. In yet another variation of the above process, deprotection of the silyl ethers to form tetra-ol 6 is performed using acetic acid, IPAc/MeOH, or acetic acid/MeOH/Water. In another variation, the acetalization of tetra-ol 6 to form compound 7 uses acrolein dimethyl acetal or the acrolein diethyl acetal analog, in DCM or toluene and TFA. In one aspect of the process, the coupling of compound 7 with compound 8a to afford compound 9a is performed with PIV-Cl, TEA, DMAP and THF. In another aspect of the process, the coupling of compound 7 with compound 8b to afford compound 9b is performed with PIV-Cl, TEA, DMAP and THF. In one variation of the process, deprotection of compound 9a to form compound 10 is performed using HCl in MeOH. In one variation, deprotection of compound 9b to form compound 10 is performed using HCl in MeOH. In certain variation of the above process, the process requires 2, 3, 4, 5 or 6 isolation steps. In one variation, the compound 2 is a mixture of compounds 2a and 2b. In another variation, the mixture of compounds 2a and 2b is used in subsequent step without isolation or purification. As used herein, the elimination of an “isolation” step of the intermediate product from a reaction mixture means that the intermediate product that is obtained in its “crude” or non-purified form, with or without the solvent in which the process was performed in, may be used in the subsequent step to provide the desired product in good yields without the need for the isolation and/or purification of the intermediate product. Such a lack of an isolation and or purification step or procedure is of significant advantage in processing cycle time, throughput and cost, especially when the reaction is performed in a production or manufacturing scale.

Depending on the desired purity of the intermediate(s) and the processing parameters that are used in the process, the intermediates described herein may be isolated and/or purified in one or more processing step before submitting to the subsequent reaction step or steps. In particular aspects of the process, depending on the desired purity, the reagents employed and the reaction conditions, the subsequent reaction step or steps of a reaction product (or intermediate) is subjected to one or more subsequent reaction without isolation and/or purification until the final product compound 10 is obtained. When desired, purification of the intermediates and/or product may be performed using various methods known in the art, including column chromatography, crystallization, distillation and the like, or the combination of the methods.

The two-step coupling reaction and hydrolysis to form compound 10 may be performed using compound 7 with various side chain acids and side chain acid salts and various selected coupling agents and reaction conditions to provide compound 10 in. high yields.

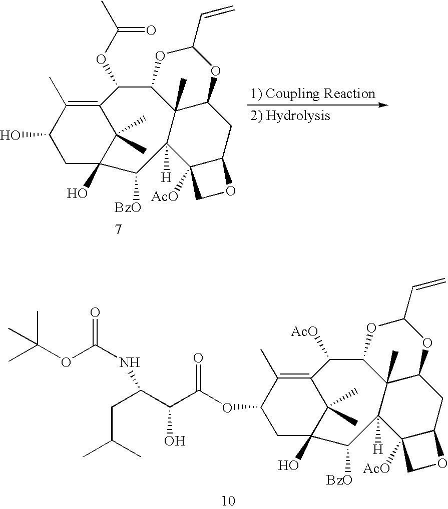

In another embodiment, there is provided a process for preparing compound 9:

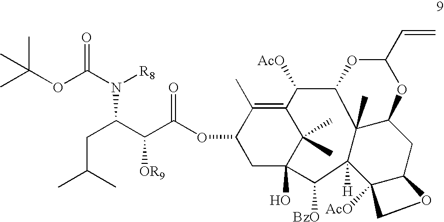

wherein R₈ and R₉ together with the nitrogen and oxygen to which they are attached form a cyclic 2,4-dimethoxy benzylidene N,O-acetal or a cyclic 2,6-dimethoxy benzylidene N,O-acetal; the process comprising contacting a compound of the formula 7 with a side chain compound 8 and a coupling reagent under a coupling condition sufficient to form compound 9;

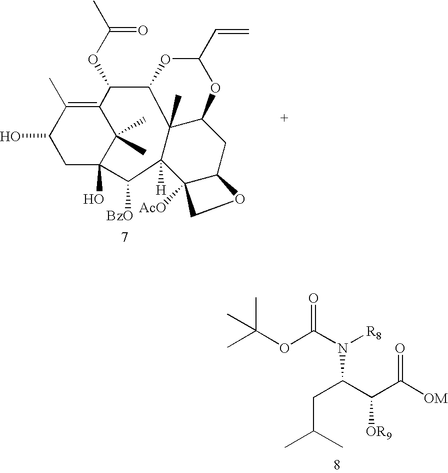

wherein R₈ and R₉ together with the nitrogen and oxygen to which they are attached form a cyclic 2,4-dimethoxy benzylidene N,O-acetal or a cyclic 2,6-dimethoxy benzylidene N,O-acetal; and M is H or an alkali metal selected from the group consisting of Li, Na and K. In a particular variation of the above process, the side chain is compound 8, wherein R₈ and R₉ together with the nitrogen and oxygen to which they are attached form a cyclic 2,4-dimethoxy benzylidene N,O-acetal or a cyclic 2,6-dimethoxy benzylidene N,O-acetal; and M is H or an alkali metal selected from the group consisting of Li, Na and K.

In one variation of the above process, the process provides the diastereoisomer compounds of the formulae 9c and 9d as a single diastereoisomer, or as a mixture of the two diastereoisomers:

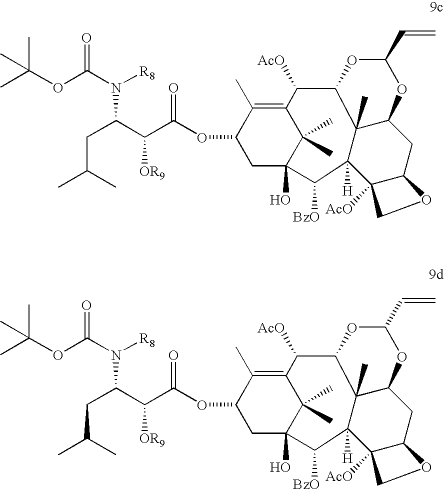

wherein R₈ and R₉ are as defined above.

In another variation, the coupling reagent is selected from the group consisting of alkyl, aryl or arylalkyl acid anhydrides; dicarbonates; alkyl, aryl or arylalkyl haloformates; alkyl, aryl or arylalkyl acid halides; chlorosulfonates, sulfonic anhydrides, alkyl, aryl, arylalkyl isocyanate. In another variation, the coupling condition comprises THF or toluene or mixtures thereof, NMM and DMAP. In yet another variation, the coupling reagent is selected from the group consisting of benzoic anhydride, phenoxyacetic anhydride, trifluoroacetic anhydride, trimethylacetic anhydride, acetic anhydride, hexanoic anhydride, benzyl chloroformate, trichloroethyl chloroformate, methyl chloroformate, 4-nitrophenyl chloroformate, benzoyl chloride, 2-methoxybenzoyl chloride, 2-chloro-2,2-diphenylbenzyl chloride, 2,4,6-trichlorobenzoyl chloride, pentafluorobenzoyl chloride, 4-nitro-benzoyl chloride, 2-chloro-benzoyl chloride, phenoxyacetyl chloride, 4-chloromethyl-benzoyl chloride, acetyl chloride, trimethyl acetyl chloride, hexanoyl chloride, trimethylacetyl chloride, methane sulfonyl chloride, p-tosyl chlorothionoformate, phenylisocyanate and p-toluenesulfonic anhydride. In yet another variation of the above process, the coupling reagent is selected from the group consisting of benzoic anhydride, 2,4,6-trichlorobenzoyl chloride and di-t-butyl dicarbonate. In another variation, the product from the coupling reaction is further hydrolyzed to form compound 10 in >90% yield, wherein R₈ and R₉ are hydrogen.

In another aspect of the above process, the deprotection (or hydrolysis) provides the compound 10a as the single diasteroisomer, the compound 10b as the single diastereoisomer, or the compound 10 as a mixture of both diastereoisomers 10a and 10b.

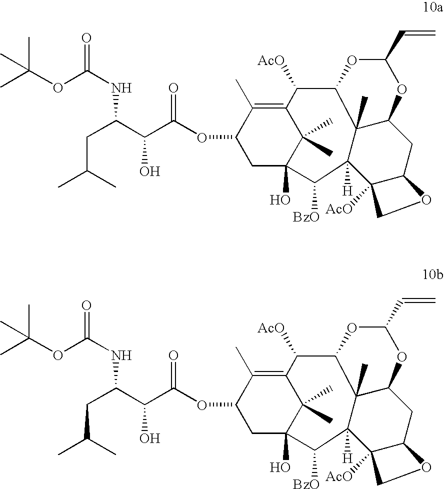

In the above process using the activated acyl coupling reaction of the side chain, the compound that is formed include:

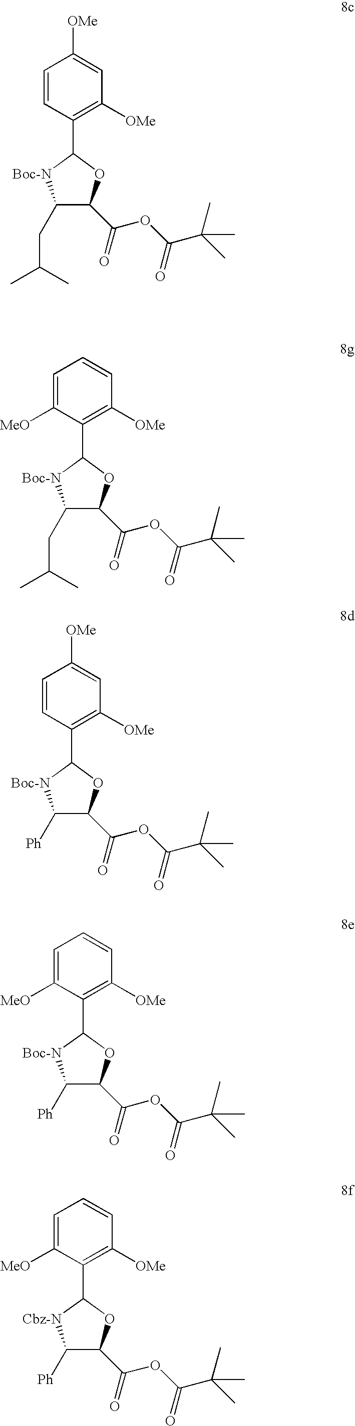

In the above process, the side chain that may be used in the coupling reaction include:

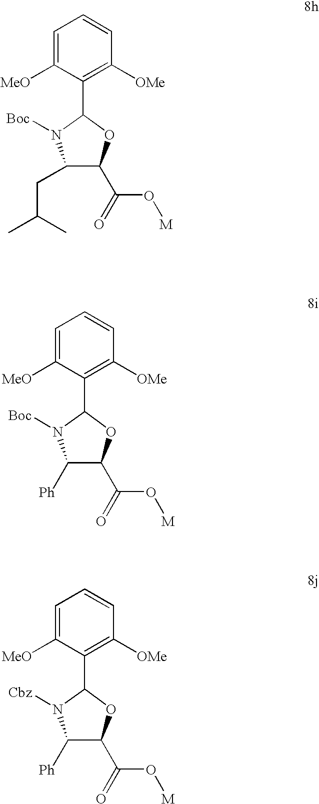

Also provided herein are the side chain compound comprising the formulae:

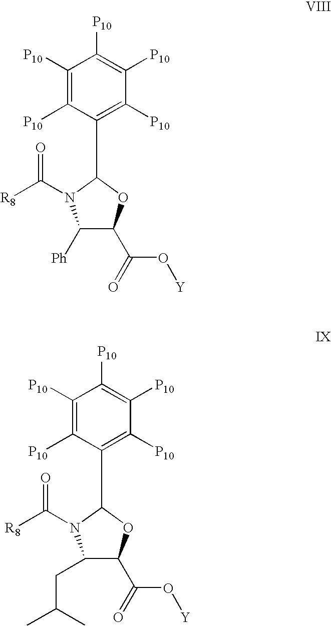

wherein:

R₈ is selected from the group consisting of C₁-C₆ alkoxy, aryloxy, C₁-C₆ alkyl, arylCH₂O—, aryl and heteroaryl;

each P₁₀ is independently H or is an electron donating or electron withdrawing substituent;

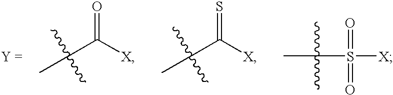

and

X is selected from the group consisting of substituted or unsubstituted C₁-C₁₂ alkyl, C₂-C₁₀ alkenyl, aryl and heteroaryl.

Non-limiting representative examples of the phenyl substituted group with P₁₀ include: phenyl, 2-methoxyphenyl, 4-methoxyphenyl, 2,4-dimethoxyphenyl, 2,6-dimethoxyphenyl, 3,4,5-trimethoxyphenyl, 4-bromophenyl and the like.

Also provided herein are the side chain compound comprising the formulae:

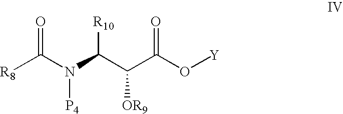

wherein:

R₈ is selected from the group consisting of C₁-C₆ alkoxy, aryloxy, C₁-C₆ alkyl, arylCH₂O—, aryl and heteroaryl;

R₉ is H or is selected from the group consisting of BOM, Bn, P₃ and a hydroxyl protecting group;

P₄ is H, or P₄ and P₃ together with the nitrogen and oxygen to which P₄ and P₃ are attached form a substituted or unsubstituted cyclic C₁-C₆ alkyl, C₂-C₁₀ alkenyl or aryl acetal, or benzylidene N,O-acetal;

R₁₀ is H or is selected from the group consisting of C₁-C₆ alkyl, C₂-C₁₀ alkenyl, aryl and heteroaryl;

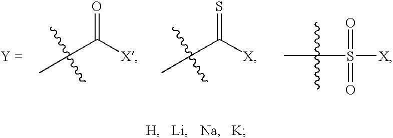

X is selected from the group consisting of substituted or unsubstituted C₁-C₁₂ alkyl, C₂-C₁₀ alkenyl, aryl and heteroaryl; and

X′ is selected from substituted or unsubstituted aryl and heteroaryl;

provided that when Y is H, Li, Na or K and when R₁₀ is isobutyl or phenyl, then P₄ and P₃ together with the nitrogen and oxygen to which P₄ and P₃ are attached are not a cyclic benzylidene N,O-acetal, a cyclic 2,4-dimethoxy benzylidene N,O-acetal, a cyclic 3,4-dimethoxy benzylidene N,O-acetal or a cyclic 4-methoxy benzylidene acetal.

In one variation of the above compound, R₉ is selected from the group consisting of BOM, Bn, P₃ and a hydroxyl protecting group. In another variation, P₄ and P₃ together with the nitrogen and oxygen to which P₄ and P₃ are attached form a substituted or unsubstituted cyclic C₁-C₆ alkyl, C₂-C₁₀ alkenyl or aryl acetal, or benzylidene N,O-acetal. In another variation of the above compound, R₁₀ is selected from the group consisting of C₁-C₆ alkyl, C₂-C₁₀ alkenyl, aryl and heteroaryl.

A process for preparing compound 20;

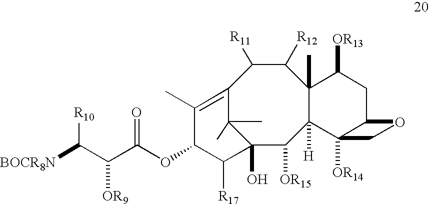

wherein:

R₈ is H or together with the nitrogen and oxygen to which they are attached form a cyclic 2,4-dimethoxy benzylidene N,O-acetal or a cyclic 2,6-dimethoxy benzylidene N,O-acetal;

[symbol]

the process, the coupling reagent is selected from the group consisting of benzoic anhydride, phenoxyacetic anhydride, trifluoroacetic anhydride, trimethylacetic anhydride, acetic anhydride, hexanoic anhydride, benzyl chloroformate, tri-chloroethyl chloroformate, methyl chloroformate, 4-nitrophenyl chloroformate, benzoyl chloride, 2-methoxybenzoyl chloride, 2-chloro-2,2-diphenylbenzoyl chloride, 2,4,6-trichlorobenzoyl chloride, pentafluorobenzoyl chloride, 4-nitro-benzoyl chloride, 2-chloro-benzoyl chloride, phenoxyacetyl chloride, 4-chloromethyl-benzoyl chloride, acetyl chloride, hexanoyl chloride, methane sulfonyl chloride, p-tosyl chlorothionoformate, phenylisocyanate and p-toluene sulfonic anhydride.

In a particular variation of the above process, the coupling reagent is selected from the group consisting of benzoic anhydride, 2,4,6-trichlorobenzoyl chloride and di-t-butyl dicarbonate. In yet another variation of the above process, the coupling product from the coupling reaction is further hydrolyzed to form compound 20 in >90% yield, wherein R₈ and R₉ are hydrogen. In another variation of the process, wherein in compound 21, R₁₁ is α-OAc—; R₁₂ is α-OR_{12′} and R₁₃ is -R_{13′}, wherein R_{12′} and R_{13′} together with the oxygen atoms to which they are attached form a cyclic allyl acetal; R₁₄ is CH₃CO; R₁₅ is PhCO; and in compound 22, M is Na; R₁₅ is H, R₉ is BOM; and R₁₀ is C₁-C₆ alkyl; to form the corresponding substituted product 20. In another variation of the process, in compound 21, R₁₁ is P₃O— wherein P₃ is CBz; R₁₂ is oxo; R₁₃ is CBz; R₁₄ is CH₃CO; R₁₅ is PhCO; and in compound 22, M is Na; R₈ is H, R₉ is BOM; and R₁₀ is C₁-C₆ alkyl; to form the corresponding substituted product 20.

In another variation of the process, in compound 21, R₁₁ is β-OAc wherein P₃ is CBz; R₁₂ is oxo; R₁₃ is CBz; R₁₄ is CH₃CO; R₁₅ is PhCO; and in compound 22, M is Na; R₈ is H, R₉ is BOM; and R₁₀ is C₁-C₆ alkyl; to form the corresponding substituted product 20.

A process for preparing compound 30:

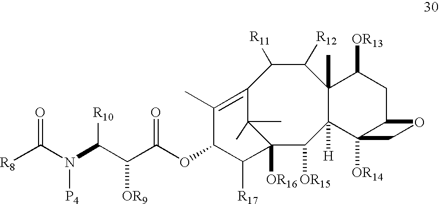

wherein:

R₈ is selected from the group consisting of C₁-C₆ alkyl, C₁-C₆ alkoxy, arylC₁-C₆ alkoxy, arylC₁-C₆ alkoxyCH₂—, aryl and heteroaryl;

R₉ is hydrogen or is selected from the group consisting of P₅, C₁-C₄ alkylCO, PhCO, arylC₁-C₃ alkyl, arylC₁-C₆ alkoxyCH₂—, TES, TMS, iPrDMS, TBDMS, MDiPrS, TBDPS and TPS;

R₁₀ is H or is selected from the group consisting of C₁-C₆ alkyl, C₂-C₁₀ alkenyl, aryl and heteroaryl;

R₁₁ is selected from the group consisting of oxo, α-OP₆, β-OP₆, C₁-C₆ alkylCOO—, arylCOO—, or P₆ and P₇ together with the oxygen atoms to which they are attached form an unsubstituted or substituted 5-membered cyclic alkyl, alkenyl or aryl acetal;

R₁₂ is selected from the group consisting of oxo, P₇O—, α-OR)_{12′}, β-OR_{12′}, C₁-C₆ alkylCOO— and arylCOO—, or P₇ and —P_{13′} together with the oxygen atoms to which they attach form an unsubstituted or substituted 6-membered cyclic alkyl, alkenyl or aryl acetal;

R₁₃ is selected from the group consisting of —P_{13′}, TES, TMS, iPrDMS, TBDMS, MDiPrS, TBDPS, TPS, a hydroxyl protecting group, or —P_{13′} and —P₇ together with the oxygen atoms to which they attach form an unsubstituted or substituted 6-membered cyclic alkyl, alkenyl or aryl acetal;

R₁₄ is selected from the group consisting of C₁-C₄ alkylCO, PhCO and R₁₈CO₂— where R₁₈ is selected from the group consisting of C₁-C₆ alkyl, C₁-C₆ alkylaryl, aryl and heteroaryl;

R₁₅ is selected from the group consisting of C₁-C₄ alkylCO, PhCO and R₁₉CO₂— where R₁₉ is selected from the group consisting of C₁-C₆ alkyl, C₁-C₆ alkylaryl, aryl and heteroaryl;

R₁₆ is hydrogen or together with R₁₇ forms a cyclic carbonate (—OCOO—) or cyclic acetal (—O—CH₂—O—);

R₁₇ is hydrogen, —OH or together with R₁₆ forms a cyclic carbonate (—OCOO—) or cyclic acetal (—O—CH₂—O—);

P₄ is hydrogen or R₉ and P₄ together with the oxygen and nitrogen atoms to which they are attached form a unsubstituted or substituted 5-membered cyclic benzylidene N,O-acetal;

P₅ is a hydroxyl protecting group;

P₆ is hydrogen or is selected form the group consisting of C₁-C₄ alkylCO, PhCO, arylC₁-C₆ alkoxyCH₂—, TES, TMS, iPrDMS, TBDMS, MDiPrS, TBDPS and TPS, a hydroxyl protecting group, or P₆ and P₇— together with the oxygen atoms to which they attach form a 5-membered cyclic alkyl, alkenyl or aryl acetal;

P₇ is hydrogen or is selected form the group consisting of C₁-C₄ alkylCO, PhCO, arylC₁-C₆ alkoxyCH₂—, TES, TMS, iPrDMS, TBDMS, MDiPrS, TBDPS and TPS;

the process comprising contacting a compound of the formula 31 with a side chain compound 32 and a coupling reagent under a coupling condition sufficient to form compound 30;

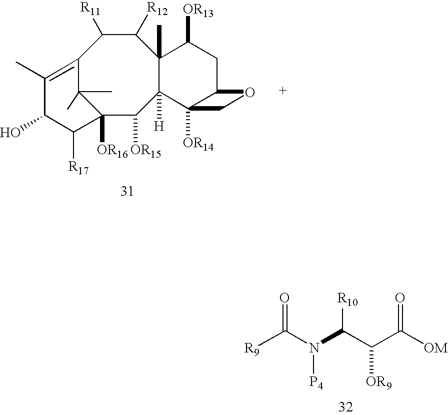

and M is H or an alkali metal selected from the group consisting of Li, Na and K. As noted herein, specific hydroxyl protecting group that may be employed in the process may include, for example, TBS, CBz, Bn, BOM, PMB, Troc, trichloroethyl, allyl, alloc, phenoxyacetate, methoxyacetate, phenylacetate, ethoxyethyl, butoxyethyl, THP other cyclic and acyclic acetals and ortho esters.

In another variation of the above process, the coupling reagent is selected from the group consisting of acid anhydrides, dicarbonates, chloroformates, acid halides, chlorosulfonates, sulfonic anhydrides, alkyl isocyanates, aryl isocyanate. In another variation, the coupling condition comprises THP or toluene or mixtures thereof, NMM and DMAP. In a particular variation of the process, the coupling reagent is selected from the group consisting of benzoic anhydride, phenoxyacetic anhydride, trifluoroacetic anhydride, trimethylacetic anhydride, acetic anhydride, hexanoic anhydride, benzyl chloroformate, trichloroethyl chloroformate, methyl chloroformate, 4-nitrophenyl chloroformate, benzoyl chloride, 2-methoxybenzoyl chloride, 2-chloro-2,2-diphenylbenzoyl chloride, 2,4,6-trichlorobenzoyl chloride, pentafluorobenzoyl chloride, 4-nitro-benzoyl chloride, 2-chlorobenzoyl chloride, phenoxyacetyl chloride, 4-chloromethyl-benzoyl chloride, acetyl chloride, trimethyl acetyl chloride, hexanoyl chloride, trimethylacetyl chloride, methanesulfonyl chloride, p-tosyl chlorothionoformate, phenylisocyanate and p-toluene sulfonic anhydride.

Equivalent protecting groups that may be used in the above cited procedures are known to one skilled in the art of organic synthesis. Such protecting groups, and the use of such groups in synthesis, may be found in various texts, including T. W. Greene and P. G. M. Wuts, Protective Groups in Organic Synthesis, 4th Edition, John Wiley & Sons, New York (2007).

Standard procedures, chemical transformation and related methods are well known to one skilled in the art, and such methods and procedures have been described, for example, in standard references such as Fiesers' Reagents for Organic Synthesis, John Wiley and Sons, New York, N.Y., 2002; Organic Reactions, vols. 1-66, John Wiley and Sons, New York, N.Y., 2005; March J. Advanced Organic Chemistry, 6^{th} ed., John Wiley and Sons, New York, N.Y.; and R. C. Larock: Comprehensive Organic Transformations, Wiley-VCH Publishers, New York, 1999. All texts and references cited herein are incorporated by reference in their entirety.

### DESCRIPTION OF THE FIGURES

FIG. 1 shows a schematic representation of one embodiment of the process for the preparation of compound 10.

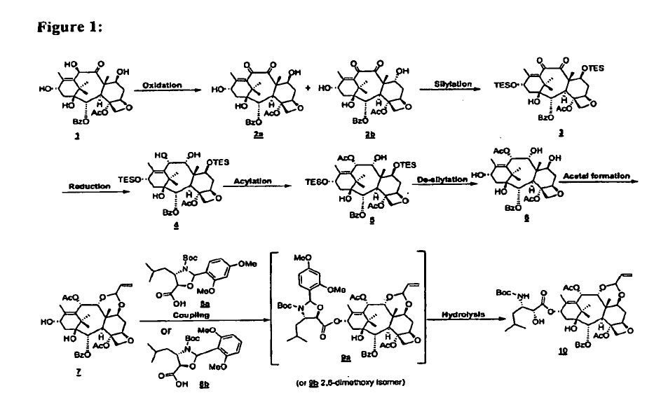

FIG. 2 shows a schematic representation of one embodiment of the process for the preparation of compound 4.

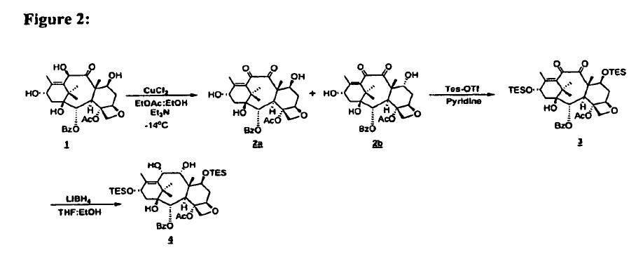

FIG. 3 shows a schematic representation of another embodiment of the process for the preparation of compound 10.

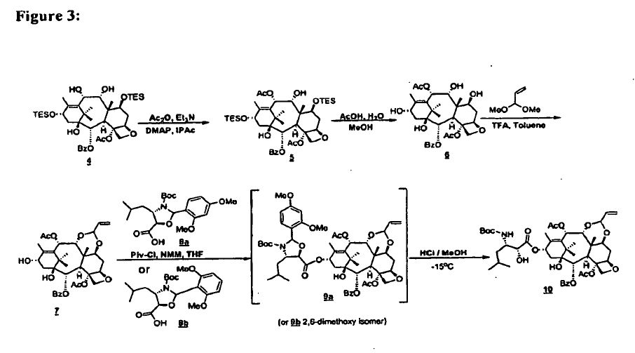

FIG. 4 shows a schematic representation of one embodiment of the process for the preparation of compound 8b.

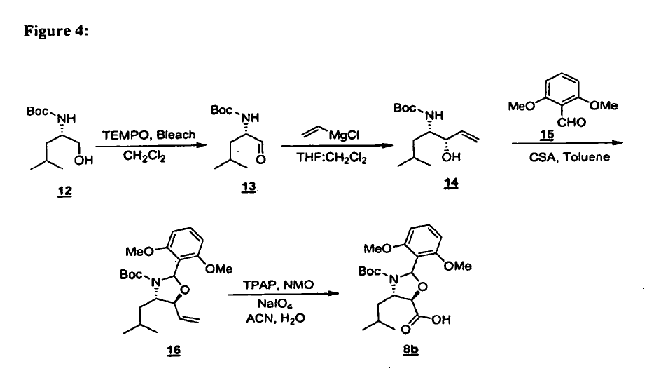

FIG. 5 shows a schematic representation of one embodiment of the process for the preparation of compound 9b.

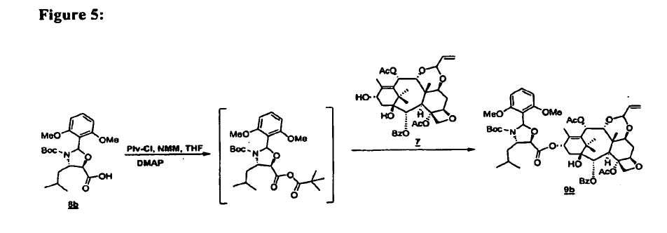

### DEFINITIONS

Unless specifically noted otherwise herein, the definition of the terms used are standard definitions employed in the art of organic synthesis and the pharmaceutical sciences.

As used herein, the term “acyl” alone or in combination, refers to an acid group in which the —OH of the carboxyl acid group is replaced by some other substituent (RCO—). Examples of such acyl group include for example, C₁-C₁₀ alkylCO—, arylCO—, such as acetyl (CH₃CO—), benzoyl (C₆H₅CO—), and the like.

The term “alkyl”, alone or in combination, refers to an optionally substituted straight-chain or branched-chain alkyl radical having from 1 to 10 carbon atoms (e.g. C₁₋₁₂ alkyl or C₁-C₂ alkyl). Examples of alkyl radicals include methyl, ethyl, n-propyl, isopropyl, n-butyl, isobutyl, sec-butyl, tert-butyl, tert-amyl, pentyl, hexyl, heptyl, octyl and the like.

The term “alkenyl”, alone or in combination, refers to an optionally substituted straight-chain or branched-chain hydrocarbon radical having one or more carbon-carbon double-bonds and having from 2 to about 18 carbon atoms. Examples of alkenyl radicals include ethenyl, propenyl, 1,4-butadienyl and the like.

The term “aryl”, alone or in combination, refers to an optionally substituted aromatic ring. The term aryl includes monocyclic aromatic rings, polyaromatic rings and polycyclic ring systems. The polyaromatic and polycyclic rings systems may contain from two to four rings, more preferably two rings. Examples of aryl groups include six-membered aromatic ring systems, including for example, phenyl, biphenyl, naphthyl and anthryl ring systems. The aryl groups of the present application generally contain from five to six carbon atoms.

The term “alkoxy” refers to an alkyl ether radical wherein the term alkyl is defined as above. Examples of alkoxy radicals include methoxy, ethoxy, n-propoxy, isopropoxy, n-butoxy, iso-butoxy, sec-butoxy, tert-butoxy and the like.

An “alpha” or “α” designation for a substituent on a molecular structure means that the substituent is attached below the plane of the paper, or shown as a dashed line.

A “beta” or “β” designation for a substituent on a molecular structure means that the substituent is attached above the plane of the paper, or shown as a wedge line.

The term “baccatin” or “baccatin derivatives” means the taxane derivatives in which the side chain at the 13-position of the taxane skeleton is a hydroxy group and these derivatives are often referred to in the literature as a baccatin or “baccatin I-VII” or the like depending, on the nature of the substituents on the tricyclic rings of the taxane skeleton.

The term “diastereoisomer” refers to any group of four or more isomers occurring in compounds containing two or more asymmetric carbon atoms. Compounds that are stereoisomers of one another, but are not enantiomers are called diastereoisomers.

“Electron donating groups” means a group or substituents that have the ability to donate electrons by an inductive effect and/or by a resonance effect. Examples of electron donating groups include —OH, —OCH₃, —OCH₂CH₃, —NH₂, —NHCH₃, alkyl groups, etc.

“Electron withdrawing groups” means a group or substituents that have the ability to withdraw electrons by an inductive effect and/or by a resonance effect. Examples of electron withdrawing groups include —NO₂, fluorine, chlorine, bromine, iodine, —COOH, —CN, etc.

“Heteroaryl” means a cyclic aromatic group with five or six ring atoms, wherein at least one ring atom is a heteroatom and the remaining are carbon atoms. Heteroaryl groups may include, for example, imidazole, isoxazole, oxazole, pyrazine, pyridine, pyrimidine, triazole and tetrazole. Heteroaryl also includes, for example, bicyclic or tricyclic heteroaryl rings. These bicyclic or tricyclic heteroaryl rings include benzo[b]furan, benzimidazole, quinazoline, quinoline, isoquinoline, naphthyridine, quinolizine, indole, indazole, benzoxazole, benzopyrazole, and indolizine. The bicyclic or tricyclic heteroaryl rings can be attached to the parent molecule through either the heteroaryl group itself or the aryl, cycloalkyl, cycloalkenyl or heterocycloalkyl group to which it is fused. The heteroaryl groups can be substituted or unsubstituted.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 4^{th} ed.; Wiley: New York, 2007). As used herein, specific hydroxyl protecting groups that may be employed in the compound disclosed in the present application include TBS, CBz, Bn, BOM, PMB, Troc, trichloroethyl, allyl, alloc, phenoxyacetate, methoxyacetate, phenylacetate, ethoxyethyl, butoxyethyl THP other cyclic and acyclic acetals and ortho esters. Exemplary silyl groups for protection of hydroxyl groups include TBDMS (tert-butyldimethylsilyl), NDMS (2-norbornyldimethylsilyl), TMS (trimethylsilyl) and TES (triethylsilyl). Exemplary NH-protecting groups include benzyloxycarbonyl, t-butoxycarbonyl and triphenylmethyl. Because of the sensitive nature of certain compounds and certain protecting groups toward hydrolysis, the judicious selection of the particular protecting group that may be used in any particular compound for any particular reaction process or processing steps. Additional, representative hydroxyl protecting groups also include acetyl, butyl, benzoyl, benzyl, benzyloxymethyl, tetrahydropyranyl, 1-ethoxyethyl, allyl, formyl and the like.

The terms “taxanes,” “taxane derivatives,” and “taxane analogs” etc. . . . are used interchangeably to mean compounds relating to a class of antitumor agents derived directly or semi-synthetically from Taxus brevifolia, the Pacific yew. Examples of such taxanes include paclitaxel and docetaxel and their natural as well as their synthetic or semi-synthetic derivatives.

The groups or functional groups described in the present application, including for example, C₁₋₁₀ alkyl, alkoxy, alkenyl, aryl, heteroaryl and the like, may be unsubstituted or may be further substituted by one or two substituents. The specific substituents may include, for example, amino, thio, halo (bromo, chloro, fluoro and iodo), oxo, hydroxyl, nitro, C₁₋₁₀ alkyl, C₁₋₁₀ alkoxy, C₁₋₁₀ alkylC(═O)— and the like.

### EXAMPLES

### I. Oxidation of 10 DAB III 1

A 4 L reaction flask, rinsed with dried EtOAc (300 mL) and held under N₂, was charged with dried EtOAc (1250 mL). Agitation was begun and dried 1 (100 g, 0.184 mol) was added. The addition of USP EtOH (800 mL) followed and the reaction mixture was cooled to −1.3° C. (internal temperature). Anhydrous CuCl₂ (86.4 g, 3.5 eq) was added and solids from the sides of the flask were washed into the mixture with anhydrous EtOH (450 mL). The reaction mixture was cooled to ≦−13° C. and anhydrous TEA (90 mL, 3.5 eq) was added slowly. The reaction was monitored by HPLC/TLC. At 1 h the reaction was judged complete (<5% 1).

TFA (36 mL) was added to quench the reaction and stirring continued for 15 min. The reaction mixture was transferred to a 10 L rotovap flask. EtOAc (500 mL) and EtOH (300 mL) were added to the reaction flask, stirred for 2 min and the rinse added to the contents of the rotovap flask, which was evaporated on the rotovap at 40° C. until no further distillation occurred (80 min). Acidified ethanol (300 mL) was added to the residue and the resulting slurry was transferred to a 2 L rotovap flask. The first rotovap flask was rinsed into the second with acidified EtOH (400 mL). Again, the mixture was evaporated on the rotovap at 40° C. until no further distillation occurred (1 h). Acidified ethanol (305 mL) was added to the rotovap flask and the mixture was stirred on the rotovap at 40° C. for 10 min. The contents of the flask were then cooled to 5° C. and filtered. The rotovap flask was rinsed (2×) with cold (2° C.) acidified ethanol (300 mL) and the rinse was transferred completely to the filter to wash the solids. The solids were dried in the vacuum oven overnight at 45° C. to give 2a. HPLC Area %=91.3%. Yield=96.72 g.

### II. Tesylation of 2a to Form 3

To 2a (96.72 g, 0.1783 mmol) in a 10 L rotovap flask was added ethyl acetate (3000 mL, 30 mL/g). The solution was evaporated on the rotovap at 40° C. to approximately half the original volume (distilled volume=1680 mL). Toluene (1000 mL, 10 mL/g) was added to the remaining solution and it was evaporated on the rotovap at 40° C. until solids were obtained (45 min). The solids were suspended in toluene (1000 mL, 10 mL/g) and the suspension was evaporated on the rotovap at 40° C. (˜1 h) to dry solids. The solids were transferred to a 2 L flask equipped with a mechanical stirrer, thermocouple, addition funnel and N₂ stream (previously purged for 5 min). The solids in the rotovap flask were rinsed into the reaction flask with anhydrous pyridine (292 mL, 3 mL/g) and agitation was begun. Upon dissolution, agitation was continued and the contents of the flask were cooled to −20° C. Triethylsilyl trifluoromethanesulfonate (120.9 mL, 3.0 eq) was slowly added to the reaction mixture to maintain the internal temperature of the reaction at ≦−10° C. After the addition of TES-OTf was complete, the reaction mixture was allowed to warm to −5.8° C. and agitation continued. Thirty minutes after the addition of TES-OTf, sampling was begun and continued at thirty-minute intervals for HPLC/TLC. The reaction was judged complete at 2 h when HPLC/TLC indicated <2% mono-TES derivative remaining.

The reaction mixture was cooled to −17.5° C. Methanol (19.3 mL, 0.2 mL/g) was added to quench the reaction and the reaction mixture was stirred for 5 min. While allowing the mixture to warm to ambient temperature, MTBE (500 mL) was slowly added with stirring and the mixture was transferred to a separatory funnel. Residues remaining in the reaction flask were washed into the separatory funnel with additional MTBE (200 mL, 2 mL/g), then water (250 mL, 2.5 mL/g) and saturated NH₄Cl solution (250 mL, 2.5 mL/g) were added. The mixture was agitated and the layers were separated. The organic layer was transferred to a clean container. MTBE (250 mL, 2 mL/g) was added to the aqueous layer. It was agitated and the layers were separated. The second organic layer was washed into the first organic layer with MTBE (100 mL) and water (200 mL, 2 mL/g) was added to the combined layers. This mixture was agitated and the layers were separated. The organic layer was transferred to a 2 L rotovap flask and evaporated to a residue at 40° C. n-Heptane (500 mL, 5 mL/g) was added to this residue and the solution was again evaporated to a residue at 40° C. n-Heptane (1000 mL, ˜10 mL/g) was added again and the solution was evaporated to one-half of its volume (distilled volume=375 mL). n-Heptane (300 mL, ˜2.5 mL/g) was added and the solution was stirred for 35 min on the rotovap at 40° C. The solution was then cooled to −15.7° C. while stirring was continued for ˜2.5 h. The solution was filtered. The solids remaining in the flask were rinsed into the filtration funnel with cold (<5° C.) n-heptane (100 mL) and all the solids were collected and dried overnight in the vacuum oven to give 111.2 g 3. HPLC Area % purity=93.4%.

### III. Reduction of 3 to Prepare 4

To a stirred solution of THF (560 mL, 5 mL/g) under N₂ in a 4 L reaction flask, was added 3 (111 g, 0.144 mol,) followed by anhydrous ethanol (560 mL, 5 mL/g). The mixture was stirred to dissolve the solids and then cooled to −12° C. 2 M LiBH₄ in THF (72 mL) was added slowly to control the reaction temperature (temp=−11.9 to −9.7° C.). The reaction mixture was stirred and sampled for HPLC/TLC at 30 min intervals. Additional 2 M LiBH₄ in THF was introduced slowly (72 mL, 1.0 eq) to the reaction flask (temp=−9.6° C. to −7.1° C.) and agitation continued for 30 min. A third addition of 2 M LiBH₄ in THF (36 mL, 0.5 eq) was made in the same manner as the previous additions (temp=−7.6° C. to −6.7° C.), but with the bath temperature adjusted to 15° C. following the addition of the LiBH₄ solution and to 12.5° C. ten minutes later. At 1 h following the final LiBH₄ addition, the reaction was judged complete (mono reduced product ≦3% relative to 4).

The reaction mixture was cooled to −10.8° C. and 10% ammonium acetate in EtOH (560 mL) was added slowly and cautiously to allow the foam to settle and to control the temperature of the solution ≦−3° C. The reaction mixture was transferred to a 2 L rotovap flask and any residues in the reaction flask were rinsed into the rotovap flask with EtOH (250 mL) and the contents of the rotovap flask were evaporated on the rotovap at 40° C. to an oil. Methanol (560 mL) was added to the residue. Water (1700 mL) was added to a 5 L flask equipped with an addition funnel and mechanical stirrer and was vigorously agitated. To precipitate the product, the methanol solution of the reaction mixture (748 mL) was slowly added to the flask containing water. The resulting mixture was filtered and the solids were washed with water (650 mL). A portion of the water was used to wash solids remaining in the precipitation flask into the filtration funnel. The solids were placed in the vacuum oven overnight at 45° C. to give 139.5 g of slightly wet non-homogeneous product, 4. HPLC area % purity=92.8%.

### IV. Acetylation/Deprotection of 4 to Prepare 6

Acetylation: To 4 (138 g, 0.178 mol) in a 2 L rotovap flask was added IPAc (1400 mL, 10 mL/g). The solution was evaporated on the rotovap at 40° C. to an oil. The procedure was repeated. Dried IPAc (550 mL) was then added to the residual oil and the contents of the rotovap flask were transferred to a 1 L reaction flask, equipped with a mechanical stirrer, addition funnel, thermocouple and a N₂ stream. The rotovap flask was washed into the reaction flask with IPAc (140 mL). DMAP (8.72 g, 0.4 eq), anhydrous TEA (170 mL, 7 eq) and acetic anhydride (100.6 mL, 6 eq) were added to the contents of the reaction flask and the mixture was stirred and heated to 35° C. While continuing agitation and heating to 35° C., the reaction was monitored by HPLC/TLC at 1-hour intervals.

Upon completion of the reaction, as indicated by the absence of 4 (3 h total time), the reaction mixture was cooled to 19.7° C. and saturated ammonium chloride solution (552 mL) was added. After stirring for 15 min, the mixture was transferred to a separatory funnel, the layers were separated and the aqueous layer was removed. Water (280 mL) was added to the organic layer and the mixture was stirred for 4 min. The layers were again separated and the aqueous layer was removed. The organic layer was transferred to a 2 L rotovap flask and the remaining content of the separatory funnel was washed into the rotovap flask with IPAc (200 mL). The mixture was evaporated to dryness on the rotovap at 40° C. to give ˜124 g 5 as pale yellow oily foam.

Deprotection: To the rotovap flask containing 5 (124 g) was added methanol (970 mL, 7 mL/g). Sampling for HPLC/TLC was begun and continued at 1-hour intervals. The 5/methanol solution was transferred to a 3 L reaction flask and agitation was begun. The remaining content of the rotovap flask was washed into the reaction flask with methanol (400 mL). Acetic acid (410 mL, 3 mL/g) and water (275 mL, 2 mL/g) were added and the reaction mixture was heated to 50° C. and stirred. With the temperature maintained between 50° C. and 55° C., the reaction was monitored by HPLC/TLC at 1-hour intervals for the disappearance of the starting material, formation and disappearance of the mono-TES intermediate and formation of the product, 6.

Upon completion (˜9 h), the reaction mixture was cooled to rt and transferred to

a 10 L rotovap flask. Solvent exchanges to n-heptane (2×1370 mL, 1×1000 mL) and IPAc (2×1370 mL, 1×1500 mL) were performed. IPAc (280 mL, 2 mL/g) and silica (140 g, 1 g/g) were added to the rotovap flask and the contents were evaporated on the rotovap at 40° C. until no further distillation occurred and free flowing solids were obtained. The dry silica mixture was loaded onto a silica pad (7 cm column, 280 g silica), conditioned with 2:1 n-heptane/IPAc (500 mL, 2 mL/g silica) and washed (4×) with 2:1 n-heptane/IPAc, 2 ml-g silica, 3400 mL total) and (4×) with 1:1 n-heptane/IPAc (3020 mL total, 2 mL/g silica) until all impurities were removed as indicated by TLC. Each wash (˜840 mL) was collected as a separate fraction and analyzed by TLC. The silica pad was then washed (5×) with waEtOAc (1% water, 1% AcOH in EtOAc) (3950 mL total, 2 mL/g silica) and with 1:1 MeOH/EtOAc and each wash (˜840 mL) was collected as a separate fraction. The product eluted with fractions 11-15. The fractions containing 6 as indicated by HPLC/TLC were combined, transferred to a rotovap flask and evaporated to dryness on the rotovap at 40° C. The residue in the flask was dissolved and evaporated to dryness: first with IPAc (1055 mL) and n-heptane (550 mL) and a second time with IPAc (830 mL) and n-heptane (410 mL). IPAc (500 mL) was then added to the residue, the solution was transferred to a 2 L round bottom flask and n-heptane (140 mL) was added. The resulting solution was evaporated on the rotovap and dried in the vacuum oven at 40° C. to give 6 as foam. To dissolve the foam, IPAc (160 mL) was added to the flask followed by toluene (800 mL). The solution was evaporated on the rotovap under vacuum at 50° C. until half of the solvent was removed and solids were forming. The contents of the flask were stirred and cooled to 21° C. for 1.5 h. The solids were filtered in a 90 cm filtration funnel on #54 Whatman filter paper and were washed with toluene (165 mL), transferred to the vacuum oven and dried at 40° C. to give 62.6 g of 6. HPLC area %=96.9%

### VI. Acetal Formation: 6 to 7

To a 3 L reaction flask containing 6 (25 g, 42.4 mmol) was added toluene (375 mL) and the reaction mixture was cooled to ˜−15° C. TFA (9.8 mL, 3.0 eq) was slowly added. This was followed by the addition of acrolein diethyl acetal (8.7 g) and the reaction was monitored by HPLC until <3% of 6 remained.

Hydrated silica was prepared by mixing silica (25 g) and water (25%) and a “basified silica” mixture was prepared by mixing a solution of K₂CO₃ (17.6 g, 3.0 eq) in water (1 mL/g 6) with 50 g silica.

Upon reaction completion, the hydrated silica was added to the reaction mixture and it was stirred for 30-45 min while maintaining the temperature ≦5° C. The basified silica was then added to the mixture while continuing to maintain the temperature ≦5° C. and the pH>5. After stirring for ˜15 min, the mixture was filtered. The silica was washed with ˜20 mL/g toluene and the filtrates were combined and concentrated. The residue was digested with 1 mL/g toluene for ˜4 h. The resultant solids were filtered and washed with 80:20 toluene/heptane to give 25 g of 7. HPLC area %=98%. Mass yield=66%.

### VII. Preparation of Compound 10 from 7

To THF (300 mL, 8 mL/g) stirring in a 1 L reaction flask (rinsed with THF (500 mL)) was added 7 (35.7 g, 0.0570 mol). Purified 8a (30.9 g, 1.25 eq) was added to the reaction mixture followed by the addition of NMM (11.5 mL, 1.8 eq), DMAP (2.77 g, 0.4 eq) and THF (75 mL, 2 mL/g). The mixture was stirred while N₂ was bubbled from the bottom of the flask to mix and dissolve the solids. Pivaloyl chloride (11.5 mL, 1.6 eq) was then added slowly to the reaction mixture. The reaction mixture was warmed and the temperature maintained at 38° C.±4° C. while stirring continued and N₂ continued to be bubbled from the bottom of the flask. The reaction mixture was analyzed by HPLC/TLC for consumption of starting material and formation of the coupled ester, 9a, at 30 min intervals beginning 30 min after the addition of the pivaloyl chloride.

After 1 h the reaction was judged complete and the reaction mixture was cooled to 2° C. 0.5 N HCl in MeOH (280 mL, ˜20 mL/mL NMM) was added to maintain the pH of the reaction mixture=1.5-1.9. The reaction mixture was stirred at 2° C.±2° C. and monitored by HPLC/TLC at 30 min intervals for consumption of 9a and formation of 10 and the acrolein acetal hydrolyzed by-product. Upon completion at 2 h the reaction was quenched with 5% aqueous sodium bicarbonate (300 mL) and IPAc (185 mL, 5 mL/g) was added. The reaction mixture was transferred to a 2 L rotovap flask and the reaction flask rinsed into the rotovap flask 2× with 60 mL IPAc. The mixture was evaporated under vacuum at 40° C. until a mixture of oil and water was obtained. IPAc (200 μL) was added to the oil and water mixture and the contents of the flask were transferred to a separatory funnel. The reaction flask was rinsed into the separatory funnel with IPAc (100 mL) and the contents of the separatory funnel were agitated and the layers were separated. The aqueous layer was removed. Water (70 mL) was added to the organic layer and, after agitation, the layers were separated and the aqueous layer was removed. The organic layer was transferred to a rotovap flask and evaporated under vacuum at 40° C. to a foam, which was dried in the vacuum oven to give 64.8 g crude 10. HPLC area %=45.5%.

### VIII. Purification Procedures

Normal Phase Chromatography: The 6″ Varian DAC column was packed with Kromasil (5 Kg, 10 μm, 100 Å normal phase silica gel). The 50-cm bed length provided a 9 L empty column volume (eCV). The column had been regenerated (1 eCV 80:20 waMTBE:MeOH) and re-equilibrated (1 eCV waMTBE, 1 eCV 65:35 n-heptane:waMTBE).

The crude 10 (64.70 g), was dissolved in MTBE (180 mL) and heated to 40° C. n-Heptane (280 mL) was slowly added to the solution. This load solution was pumped onto the column using a FMI “Q” pump. The column was then eluted with 65:35 n-heptane:waMTBE at 800 mL/min. A 34 L forerun (˜3.8eCV) was collected followed by 24 fractions (500 mL each). Fractions 1 through 23 were combined and concentrated to dryness on a rotovapor. The residue was dried in the vacuum oven overnight to provide 41.74 g 10. HPLC area %=99.4%.

Final Purification: The normal phase pool was dissolved in USP EtOH (6 mL/g) and concentrated to dryness three times. The resultant residue was dissolved in USP EtOH (2 mL/g). This ethanolic solution was slowly added dropwise to water (deionized, 20 mL/g) with vigorous stirring. The resultant solids were vacuum filtered and washed with cold DI water. The solids were dried in the vacuum oven at 40° C. overnight to give 38.85 g 10. HPLC area %=99.5%.

### IX. Preparation of 8b

In a round bottom flask, NMO (10.5 g, 75.2 mmol) was stirred with ACN (200 mL) to obtain a solution. With stirring, to the solution was added 10% aqueous NaIO₄ (165 mL, 76.4 mmol), additional ACN (50 mL) and deionized water (56 mL). TPAP (504 mg, 1.4 mmol) was added after which a solution 16 (15.0 g, 38.3 mmol, 0.5 g/mL ACN) was added over the course of approximately 1 minute under ambient conditions. After ˜50 minutes additional ACN (50 mL), NMO (10.0 g, 71.7 mmol) and 10% aqueous NaIO₄ (82 mL, 38.0 mmol) were added to the reaction mixture to drive to completion. After reaction was completed, to the stirring reaction mixture was added IPAc (300 mL) and water (200 mL). The mixture was vacuum filtered to remove precipitated reagents, and then it was partitioned. The aqueous phase was twice back extracted, once with IPAc and then with 2:1 r-heptane/IPAc. After each extraction the organic phases were combined.

After ensuring that the organic phase was slightly acidic, it was washed with 15% aqueous Na₂S₂O₃, followed by water and finally brine. The isolated organic phase was concentrated by rotary evaporation at 45° C. to give 9.82 g of crude 8b. The crude oil was purified by column chromatography to give 5.0 g of 8b.

### X. Coupling of 8b to 7 to Form 10

Anhydride/Coupling 8b with 7:

A 10 mL round bottom flask with two necks was heated to eliminate water, then allowed to cool under N₂ atmosphere. To the flask was added 7 (125 mg, 0.2 mmol), THF (1.25 mL), 4-methylmorpholine (40 μL, 0.36 mmol), DMAP (10.9 mg, 0.009 mmol), 8b sodium salt (110 mg, 0.254 mmol) and finally trimethylacetyl chloride (40 μL, 0.319 mmol). The reaction mixture was stirred at 40° C. under N₂. After about 2 hours, additional 4-methylmorpholine (11 μL, 0.01 mmol), 8b sodium salt (41 mg, 0.1 mmol) and trimethylacetyl chloride (13 μL, 0.1 mmol) were added to assist formation of the anhydride intermediate which then coupled to 7. After about 2 additional hours, 4-methylmorpholine (11 μL, 0.010 mmol), trimethylacetyl chloride (13 μL, 0.104 mmol) and 8b sodium salt (42 mg, 0.1 mmol) were added. After 1.5 hours more, the reaction was placed into a freezer at −20° C. overnight. The following morning, stirring was resumed and the reaction was heated to 45° C. for 2 hours. Additional 4-methylmorpholine (22 μL, 0.02 mmol) and trimethylacetyl chloride (25 μL, 0.201 mmol) were added. An additional 2 hours of stirring resulted in the reaction reaching ˜90% completion.

To quench, the reaction mixture was removed from heat and allowed to cool to RT with stirring, and MTBE (2 mL) was added followed by water (1 mL). The mixture was partitioned and the organic phase was washed with brine (40 μL). The organic phase was concentrated at 40° C. to obtain crude product as a pink foam.

The pink foam was dissolved into MTBE (500 μL) and added dropwise to stirring n-heptane (5 mL) at ˜20° C. to give pink precipitate. The mixture was vacuum filtered and the solids were dried overnight in a vacuum oven at 40° C. to yield the desired coupled ester (82 mg), as indicated by LC/MS. The coupled ester 9b was purified by flash chromatography on normal phase silica, eluting with an IPAc/n-heptane system of increasing polarity. Approximately 26 mg of the purified coupled ester 9b was recovered as confirmed by LC/MS.

### Deprotection of 9b to Form 10:

The coupled ester 9b (15 mg, 0.001 mmol) was dissolved into THF (1 mL). A 250 μL aliquot of the solution was diluted 1:1 with THF. The solution was stirred on an ice bath at ˜0° C., after which HCl (0.5 N in MeOH, 25 μL) was added. The reaction was monitored by LC/MS, which indicated the formation of 10.

### VIII. Coupling Reactions: Preparation of Compound 10 from 7

Coupling of Compound 7 to Compound II Using Variety of Coupling

Reagents:

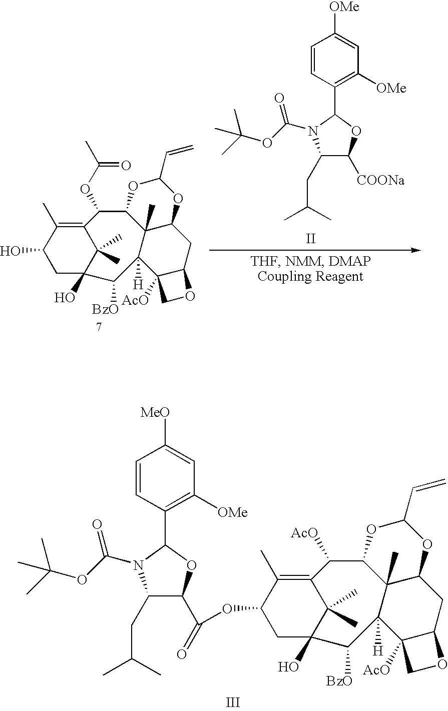

To each of four reaction flasks containing 7 (100 mg) and II (120 mg, 1.75 eq) were added anhydrous THF (2 mL) followed by DMAP (8 mg, 0.4 eq) and NMM (44 μL, 2.0 eq). As shown in Table 1, to each of these four reaction mixtures was added a different coupling reagent (2.0 eq). The reaction mixtures were stirred at rt for 1.5 h when reactions A, B and C were judged complete by HPLC analysis. Each of these reaction mixtures was transferred to a 5 mL volumetric flask and diluted with THF. A sample of each of the solutions (25 μL) was removed, diluted with 1 mL ACN and 0.5 μL was injected on the HPLC-MS. The yield was calculated against a coupled ester external standard.

After 1.5 h, 7 was still present in Reaction D. Additional II (1.75 eq), NMM (2.5 eq) and benzyl chloroformate (6.0 eq) were introduced to the reaction mixture and the reaction continued to proceed. The major impurity that was generated had a mass consistent with the benzyl ester of the side chain. After stirring overnight, Reaction D was transferred to a 5 mL volumetric flask and sampled as above. The yield results of the four reactions are summarized in Table 1.

In certain procedures employing the above methods, the side chain II may be the sodium salt, an alkali metal salt including, for example, Li or K, or the side chain II may be a carboxylic acid (i.e., —COOH).

TABLE 1
Yields of Coupling With Representative Coupling Reagents

| Reaction | Coupling Reagents | Yield |
|---|---|---|
| A | Benzoic anhydride | 100% |
| B | 2,4,6-Trichlorobenzoyl | 100% |
| | chloride | |
| C | di-t-Butyldicarbonate | 87% |
| D | Benzyl chloroformate | 28% |

Coupling of Compound 7 with Side Chain II with Coupling Reagents:

Additional reactions with alternate coupling reagents were performed. The reactions employed compound 7 at a 50 mg scale. The reaction mixtures were sampled and the reaction volumes were estimated for yield calculations against a coupled ester external standard. No additional efforts were expended to drive the reactions to completion. The coupling reagents that provided the coupled ester III are reported in Table 2.

TABLE 2
Coupling Reagents Providing Coupled Product III
Coupling Reagents

| Acid | | | Other Coupling |
|---|---|---|---|
| Anhydrides | Chloroformates | Acid Halides | Reagents |
| Benzoic | Benzyl | Benzoyl chloride | methane sulfonyl |
| anhydride | chloroformate | | chloride |
| Phenoxyacetic | Tri-chloroethyl | 2-Chloro-2,2- | p-tosyl |
| anhydride | chloroformate | diphenylbenzoyl | chlorothiono- |
| | | chloride | formate |
| Trifluoroacetic | Methyl | 2,4,6- | Phenylisocyanate |
| anhydride | chloroformate | Trichlorobenzoyl | |
| | | chloride | |
| | 4-Nitrophenyl | Pentafluorobenzoyl | p-Toluene |
| | chloroformate | chloride | sulfonic |
| | | | anhydride |
| Trimethylacetic | | 4-Nitro-benzoyl | Di-tert-butyl |
| anhydride | | chloride | dicarbonate |
| Acetic | | 2-Chloro-benzoyl | |
| anhydride | | chloride | |
| Hexanoic | | Phenoxyacetyl | |
| anhydride | | chloride | |
| | | 2-Methoxybenzoyl | |
| | | chloride | |
| | | 4-Chloromethyl- | |
| | | benzoyl chloride | |
| | | Acetyl chloride | |
| | | Hexanoyl chloride | |

Coupling of Compound 7 with Side Chain 8b:

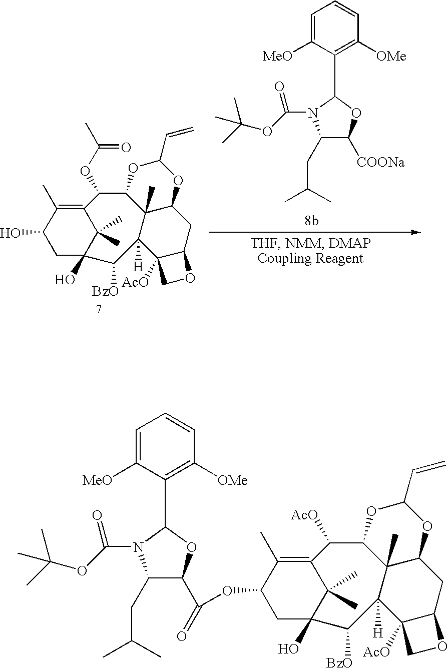

The coupling of side chain 8b using trimethyl acetyl chloride and in-situ deprotection successfully produced compound 10. To ensure that side chain 8b performed similarly to side chain II, additional tests with alternate coupling reagents were conducted. These test reactions were performed with compound 7 at a 50 mg scale. The reaction conditions and sample preparations were the same as in the previous coupling reactions. The results of the coupling of compound 7 with side chain 8b are reported in Table 3.

TABLE 3
Coupling of 7 with Side Chain 8b

| Reaction | Coupling Reagent | Yield |
|---|---|---|
| E | Benzoic anhydride | 100% |
| F | Trifluoroacetic anhydride | 100% |
| G | Benzoyl chloride | 100% |

Coupling of Compound 7 to side Chain V

The success of the coupling methodology demonstrated with a variety of choices for the coupling reagent led to investigation of the use of other protected side chains in the coupling reaction. One of these, side chain V, has the 2′-OH group protected with BOM.

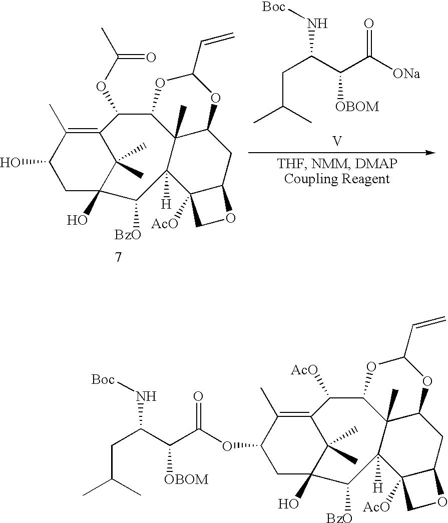

To each of two reaction flasks containing side chain V (sodium salt, 1.75 eq) in anhydrous THF (1 mL), were added DMAP (4 mg, 0.4 eq) and NMM (16 μL, 1.8 eq). Compound 7 (50 mg) was added as a solution in toluene (1 mL) to the reaction mixture. To Reaction H was added trimethylacetyl chloride (18 μL, 1.8 eq) and to Reaction J, benzoic anhydride (33 mg, 1.8 eq). The reaction flasks were transferred to a water bath (80° C.), to remove the THF. Following the removal of THF, additional trimethylacetyl chloride (1.8 eq) was introduced to Reaction H and additional NMM (1.8 eq) was introduced to both reaction mixtures. The reaction mixtures were cooled to rt and stirred overnight.

Reaction H (trimethylacetyl chloride) showed complete consumption of compound 7 and Reaction J (benzoic anhydride) showed ˜90% consumption of compound 7. The product formed during Reaction J showed a mass peak consistent with that of the coupled ester. There was no evidence of the formation of the 2′-epi isomer of the ester product.

Coupling of Side Chain V with Compound VI:

The results of the previous reactions (Reaction H and Reaction D prompted the coupling reaction of VI with V.

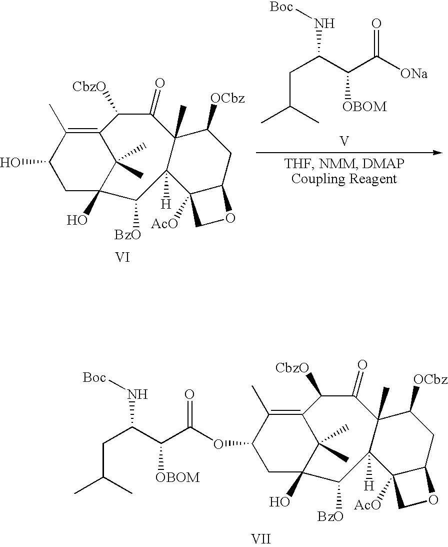

Three reactions were executed using three variants of the coupling reagent: benzoic anhydride (Reaction K), 2,4,6 trichlorobenzoyl chloride (Reaction L) and trimethyl acetyl chloride (Reaction M. The reactions were performed in toluene at room temperature. No additional efforts were expended to drive the reactions to completion. There was no evidence of the formation of 2′-epimer VII. The results of these reactions are reported in Table 4. The results reveal that acid anhydrides and acid chlorides may be used effectively as the coupling reagents in the coupling reactions of various side chains with different 13-OH baccatin derivatives.

TABLE 4
Coupling of Side Chain V with Compound VI

| Reaction | Reagent X | Yield |
|---|---|---|
| K | Benzoic anhydride | 40% |
| L | 2,4,6,Trichloroacetic anhydride | 6% |
| M | Trimethyl acetyl chloride | 12% |

What is claimed:

1. A process for preparing a 9,10-di-ketobaccatin derivative 2: 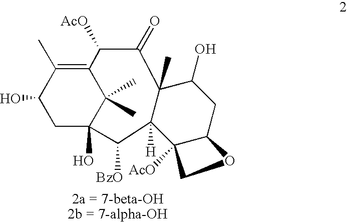
the process comprising contacting a 9-keto alcohol 1 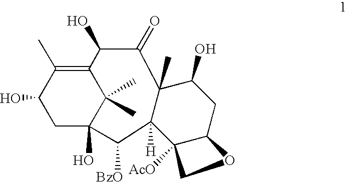
in the presence of CuCl₂ and a base.

2. The process of claim 1, wherein the base is an amine base.

3. The process of claim 2, wherein the amine base is TEA, and the process is carried out in an organic solvent or a mixture of solvents.

4. The process of claim 3, wherein the mixture of solvents comprises EtOH and EtOAc, and the process is carried out below room temperatures to form the desired product 2 in less than 3 hours.

5. The process claim 1 to afford a mixture of 2a:2b in ratio of at least 95:5 and at least 85% yield.

6. A process for the preparation of a 9,10-di-ol baccatin derivative 4: 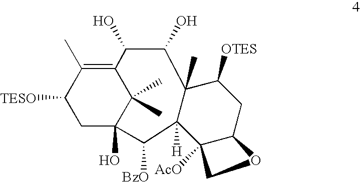
the process comprising contacting a 9,10-di-keto baccatin derivative 2 as a mixture of 2a and 2b, or 2a as a single isomer; 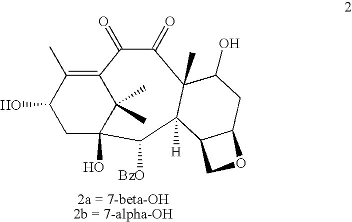
with a silylation reagent to form a di-silyl ether 3; and 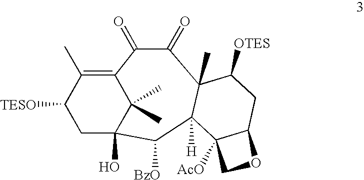
reducing the 9,10-di-ketone of the di-silyl ether 3 with a reducing reagent to form the 9,10-di-ol baccatin derivative 4.

7. The process of claim 6, wherein the silylation reagent is TES-OTf, and NMP in pyridine and the di-silyl ether 3 is formed in at least 97%.

8. The process of claim 6, wherein the reducing reagent is LiBH₄ and the reduction reaction is performed in THF/ethanol solvent to provide the 9,10-di-ol 4 in >90% yield.

9. A process for the preparation of an allylidene acetal baccatin derivative 7: 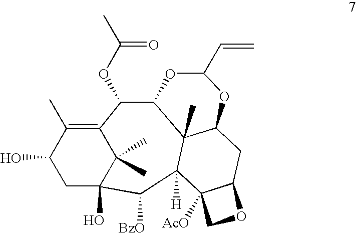
the process comprising contacting a di-ol 4 with an acylating reagent to form a 10-acylated alcohol 5; 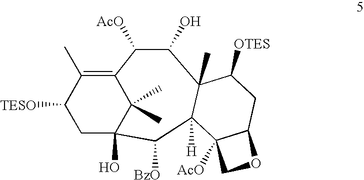
selective hydrolysis of the TES groups to form the corresponding tetra-ol 6; and 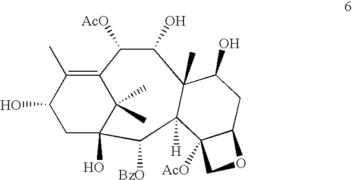
acetalization of the 7,9-di-ol of compound 6, to provide the allylidene acetal 7.

10. The process of claim 9, wherein the acylation reagent is Ac₂O, TEA and DMAP in IPAC, and the selective hydrolysis is performed with acetic acid in aqueous methanol.

11. The process of claim 9, wherein acetalization of the 7,9-di-ol of compound 6 is performed with acrolein diethyl acetal or acrolein dimethyl acetal in a non-polar solvent to provide the allylidene acetal 7, and an acid selected from the group consisting of TFA, TFA/TFAA, CSA and CDSA.

12. The process of claim 9, wherein the selective hydrolysis and the acetalization reaction steps are performed to provide the allylidene acetal 7 without isolation of compound 6.

13. The process of claim 9, wherein the selective acylation and selective hydrolysis are performed to form the tetra-ol 6 without isolation of the intermediate compound 5.

14. A compound comprising a 10-acylated alcohol 5 of the formula:

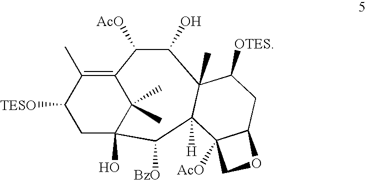

15. A compound comprising a tetra-ol 6 of the formula:

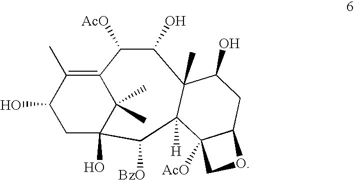

16. A compound comprising allylidene acetal 7 of the formulae:

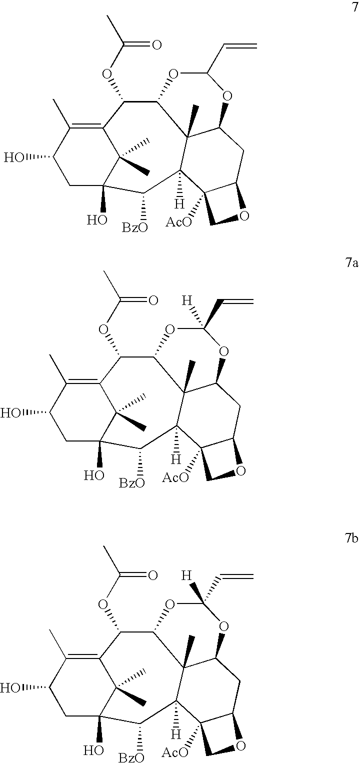

17. A process for the preparation of compound 10: 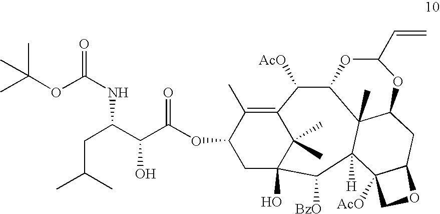
the process comprising contacting an allylidene acetal 7 with a side chain 8 under a coupling reaction condition to form a coupled intermediate compound 9; 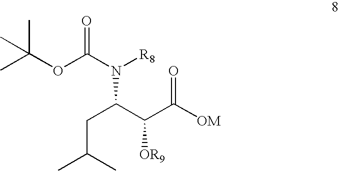
wherein R₈ and R₉ together with the nitrogen and oxygen to which they are attached form a cyclic 2,4-dimethoxy benzylidene N,O-acetal or a cyclic 2,6-dimethoxy benzylidene N,O-acetal, and M is H or an alkali metal selected from the group consisting of Li, Na and K; and 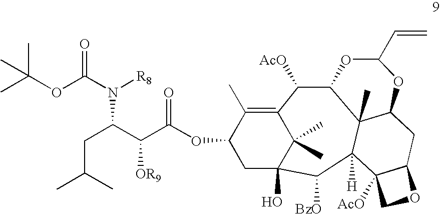
hydrolyzing compound 2 to form compound 10.

18. The process of claim 17 wherein the coupling reaction condition comprises contacting the allylidene acetal 7 with the side chain 8 in Piv-Cl, TEA, DMAP and THF or Piv-Cl, NMM, DMAP and THF for a sufficient amount of time to form compound 2 which is hydrolyzed to form compound 10 in >95% yield.

19. A compound comprising the formula 9: 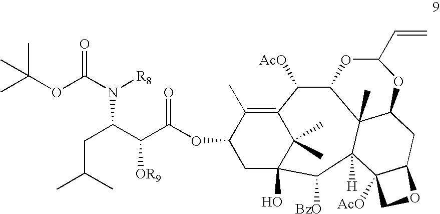
wherein R₈ and R₉ together with the nitrogen and oxygen to which they are attached form a cyclic 2,4-dimethoxy benzylidene N,O-acetal or a cyclic 2,6-dimethoxy benzylidene N,O-acetal, or where R₈ is hydrogen and R₉ is a hydroxy protecting group.

20. A process for preparing compound 9: 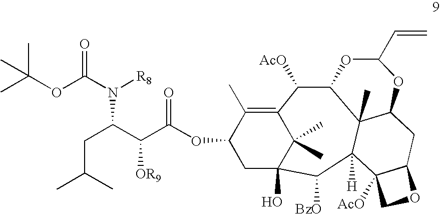
wherein R₈ and R₉ are hydrogen or together with the nitrogen and oxygen to which they are attached form a cyclic 2,4-dimethoxy benzylidene N,O-acetal or a cyclic 2,6-dimethoxy benzylidene N,O-acetal;
the process comprising contacting a compound of the formula 7 with a side chain compound 8 and a coupling reagent under a coupling condition sufficient to form compound 9; 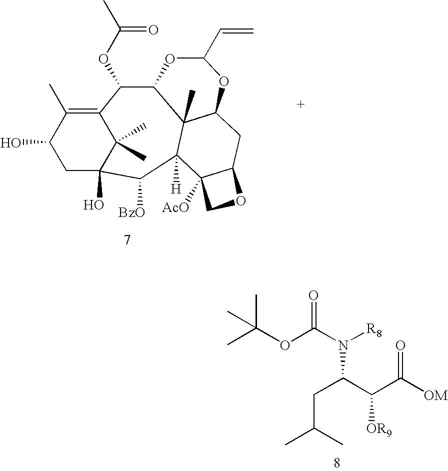
wherein R₈ and R₉ together with the nitrogen and oxygen to which they are attached form a cyclic 2,4-dimethoxy benzylidene N,O-acetal or a cyclic 2,6-dimethoxy benzylidene N,O-acetal; and M is H or an alkali metal selected from the group consisting of Li, Na and K.

21. The process of claim 20, wherein compound 9 is further hydrolyzed to form compound 10:

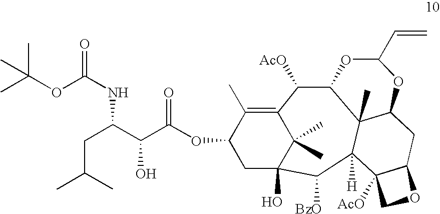

22. The process of claim 21, where the hydrolyzed compound is compound 10a:

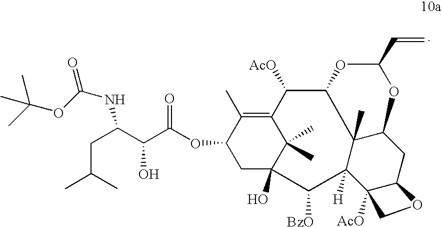

23. The process of claim 21, where the hydrolyzed compound is compound 10b.

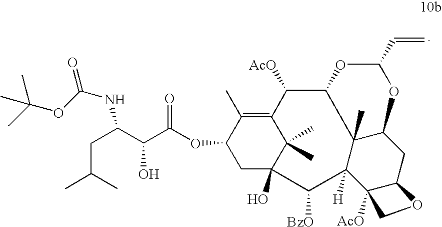

24. The process of claim 20, wherein the side chain is compound 8
R₈ and R₉ together with the nitrogen and oxygen to which they are attached form a cyclic 2,4-dimethoxy benzylidene N,O-acetal or a cyclic 2,6-dimethoxy benzylidene N,O-acetal; and
M is H or an alkali metal selected from the group consisting of Li, Na and K.

25. The process of claim 20, wherein the coupling reagent is selected from the group consisting of alkyl, aryl or arylalkyl acid anhydrides; dicarbonates; alkyl, aryl or arylalkyl chloroformates; alkyl, aryl or arylalkyl acid halides; chlorosulfonates, alkyl or aryl sulfonic anhydrides; alkyl isocyanates; alkyl, aryl or arylalkyl isocyanate; and sulfonic anhydrides.

26. The process of claim 25, wherein the coupling condition comprises THF or toluene or mixtures thereof, and NMM and DMAP.

27. The process of claim 25, wherein the coupling reagent is selected from the group consisting of benzoic anhydride, phenoxyacetic anhydride, trifluoroacetic anhydride, trimethylacetic anhydride, acetic anhydride, hexanoic anhydride, benzyl chloroformate, tri-chloroethyl chloroformate, methyl chloroformate, 4-nitrophenyl chloroformate, benzoyl chloride, 2-methoxybenzoyl chloride, 2-chloro-2,2-diphenylbenzoyl chloride, 2,4,6-trichlorobenzoyl chloride, pentafluorobenzoyl chloride, 4-nitro-benzoyl chloride, 2-chloro-benzoyl chloride, phenoxyacetyl chloride, 4-chloromethyl-benzoyl chloride, acetyl chloride, trimethylacetyl chloride, hexanoyl chloride, trimethylacetyl chloride, methane sulfonyl chloride, p-tosyl chlorothionoformate, phenylisocyanate and p-toluene sulfonic anhydride.

28. The process of claim 25, wherein the coupling reagent is selected from the group consisting of benzoic anhydride, 2,4,6-trichlorobenzoyl chloride and di-t-butyl dicarbonate.

29. The process of claim 25, wherein the product from the coupling reaction is further hydrolyzed to form compound 10 in >90% yield, wherein R₈ and R₉ are hydrogen.

30. A process for preparing compound 20: 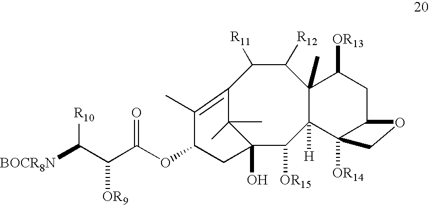
wherein:
R₈ is H or together with the nitrogen and oxygen to which they are attached form a cyclic 2,4-dimethoxy benzylidene N,O-acetal or a cyclic 2,6-dimethoxy benzylidene N,O-acetal;
R₉ is H or is selected from the group consisting of BOM, Bn and hydroxyl protecting groups;
R₁₀ is H, CO—C₆ alkyl, C₂-C₁₀ alkenyl or aryl;
R₁₁ is oxo, P₃O—, C₁-C₆ alkylCOO— or arylCOO—;
R₁₂ is oxo, α-OR_{12′}, β-OR_{12′}, C₁-C₆ alkylCOO— or arylCOO—;
R₁₃ is selected from the group consisting of -R_{13′}, α-OP₃, β-OP₃, TES, TMS, iPrDMS, TBDMS, MDiPrS, TBDPS, TPS and Bn;
R₁₄ is C₁-C₆ alkylCO or PhCO; —CO₂CH₃, C₁-C₆ alkylCO;
R₁₅ is C₁-C₆ alkylCO or PhCO;
where R_{12′} and R_{13′} together with the oxygen atoms to which they are attached form a cyclic C₁-C₆ alkyl acetal, a cyclic C₂-C₁₀ alkenyl acetal or a cyclic aryl acetal;
each P₃ is independently a hydroxyl protecting group;
the process comprising contacting a compound of the formula 21 with a side chain compound 22 and a coupling reagent under a coupling condition sufficient to form compound 20; 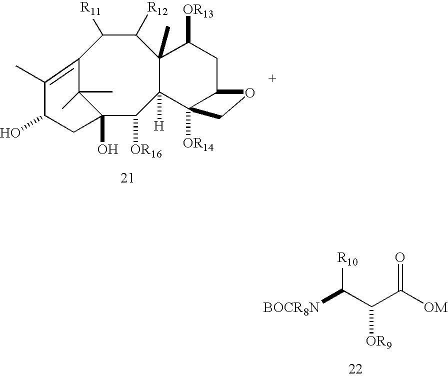
and M is H or an alkali metal selected from the group consisting of Li, Na and K.

31. The process of claim 30, wherein the side chain is compound 22;
R₈ and R₉ together with the nitrogen and oxygen to which they are attached form a cyclic 2,4-dimethoxy benzylidene N,O-acetal or a cyclic 2,6-dimethoxy benzylidene N,O-acetal;
and M is H or is Na or K; and R₁₀ is C₁-C₆ alkyl or phenyl.

32. The process of claim 30, wherein the coupling reagent is selected from the group consisting of acid anhydrides, dicarbonates, chloroformates, acid halides, chlorosulfonates, sulfonic anhydrides, alkyl isocyanates and aryl isocyanates.

33. The process of claim 30, wherein the coupling condition comprises THF or toluene or mixtures thereof, and NMM and DMAP.

34. The process of claim 30, wherein the coupling reagent is selected from the group consisting of benzoic anhydride, phenoxyacetic anhydride, trifluoroacetic anhydride, trimethylacetic anhydride, acetic anhydride, hexanoic anhydride, benzyl chloroformate, trichloroethyl chloroformate, methyl chloroformate, 4-nitrophenyl chloroformate, benzoyl chloride, 2-methoxybenzoyl chloride, 2-chloro-2,2-diphenylbenzoyl chloride, 2,4,6-trichlorobenzoyl chloride, pentafluorobenzoyl chloride, 4-nitro-benzoyl chloride, 2-chloro-benzoyl chloride, phenoxyacetyl chloride, 4-chloromethyl-benzoyl chloride, acetyl chloride, trimethylacetyl chloride, hexanoyl chloride, trimethylacetyl chloride, methane sulfonyl chloride, p-tosyl chlorothionoformate, phenylisocyanate and p-toluene sulfonic anhydride.

35. The process of claim 30, wherein the coupling reagent is selected from the group consisting of benzoic anhydride, 2,4,6-trichlorobenzoyl chloride and di-t-butyl dicarbonate.

36. The process of claim 30, wherein the coupling product from the coupling reaction is further hydrolyzed to form compound 20 in >95% yield, wherein R₈ and R₉ are hydrogen.

37. The process of claim 30, wherein:
in compound 21, R₁₁ is α-OAc—; R₁₂ is α-OR_{12′} and R₁₃ is -R_{13′}, wherein R_{12′} and R_{13′} together with the oxygen atoms to which they are attached form a cyclic allyl acetal; R₁₄ is CH₃CO; R₁₅ is PhCO; and
in compound 22, M is Na; R₈ is H, R₉ is BOM; and R₁₀ is C₁-C₆ alkyl;
to form the corresponding substituted product 20.

38. The process of claim 30, wherein:
in compound 21, R₁₁ is P₃O— wherein P₃ is CBz; R₁₂ is oxo; R₁₃ is CBz; R₁₄ is CH₃CO; R₁₅ is PhCO; and
in compound 22, M is Na; R₈ is H, R₉ is BOM; and R₁₀ is C₁-C₆ alkyl or aryl, to form the corresponding substituted product 20.

39. The process of claim 30, wherein:
in compound 21, R₁₁, is β-OAc; R₁₂ is oxo; R₁₃ is CBz; R₁₄ is CH₃CO; R₁₅ is PhCO; and
in compound 22, M is Na; R₈ is H, R₉ is BOM; and R₁₀ is C₁-C₆ alkyl or aryl to form the corresponding substituted product 20.

40. A process for preparing compound 30: 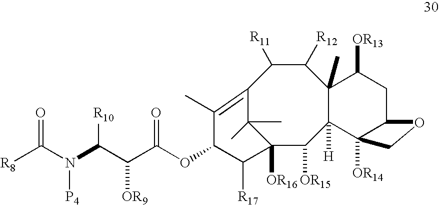
wherein:
R₈ is selected from the group consisting of C₁-C₆ alkyl, C₁-C₆ alkoxy, arylC₁-C₆ alkoxy, arylC₁-C₆ alkoxyCH₂—, aryl and heteroaryl;
R₉ is hydrogen or is selected from the group consisting of P₅, C₁-C₄ alkylCO, PhCO, arylC₁-C₃ alkyl, arylC₁-C₆ alkoxyCH₂—, TES, TMS, iPrDMS, TBDMS, MDiPrS, TBDPS and TPS;
R₁₀ is H or is selected from the group consisting of C₁-C₆ alkyl, C₂-C₁₀ alkenyl, aryl and heteroaryl;
R₁₁ is selected from the group consisting of oxo, α-OP₆, β-OP₆, C₁-C₆ alkylCOO—, arylCOO—, or P₆ and P₇ together with the oxygen atoms to which they are attached form an unsubstituted or substituted 5-membered cyclic alkyl, alkenyl or aryl acetal;
R₁₂ is selected from the group consisting of oxo, P₇O—, α-OR_{12′}, β-OR_{12′}, C₁-C₆ alkylCOO— and arylCOO—, or P₇— and —P_{13′} together with the oxygen atoms to which they attach form an unsubstituted or substituted 6-membered cyclic alkyl, alkenyl or aryl acetal;
R₁₃ is selected from the group consisting of —P_{13′}, TES, TMS, iPrDMS, TBDMS, MDiPrS, TBDPS, TPS, a hydroxyl protecting group, or —P_{13′} and —P₇ together with the oxygen atoms to which they attach form an unsubstituted or substituted 6-membered cyclic alkyl, alkenyl or aryl acetal;
R₁₄ is selected from the group consisting of C₁-C₄ alkylCO, PhCO and R₁₈CO₂— where R₁₈ is selected from the group consisting of C₁-C₆ alkyl, C₁-C₆ alkylaryl, aryl and heteroaryl;
R₁₅ is selected from the group consisting of C₁-C₄ alkylCO, PhCO and R₁₉CO₂— where R₁₉ is selected from the group consisting of C₁-C₆ alkyl, C₁-C₆ alkylaryl, aryl and heteroaryl;
R₁₆ is hydrogen or together with R₁₇ forms a cyclic carbonate (—OCOO—) or cyclic acetal (—O—CH₂—O—);
R₁₇ is hydrogen, —OH or together with R₁₆ forms a cyclic carbonate (—OCOO—) or cyclic acetal (—O—CH₂—O—);
P₄ is hydrogen or R₉ and P₄ together with the oxygen and nitrogen atoms to which they are attached form a unsubstituted or substituted 5-membered cyclic benzylidene N,O-acetal;
P₅ is a hydroxyl protecting group;
P₆ is hydrogen or is selected form the group consisting of C₁-C₄ alkylCO, PhCO, arylC₁-C₆ alkoxyCH₂—, TES, TMS, iPrDMS, TBDMS, MDiPrS, TBDPS and TPS, a hydroxyl protecting group, or P₆ and P₇— together with the oxygen atoms to which they attach form a 5-membered cyclic alkyl, alkenyl or aryl acetal;
P₇ is hydrogen or is selected form the group consisting of C₁-C₄ alkylCO, PhCO, arylC₁-C₆ alkoxyCH₂—, TES, TMS, iPrDMS, TBDMS, MDiPrS, TBDPS and TPS;
the process comprising contacting a compound of the formula 31 with a side chain compound 32 and a coupling reagent under a coupling condition sufficient to form compound 30; 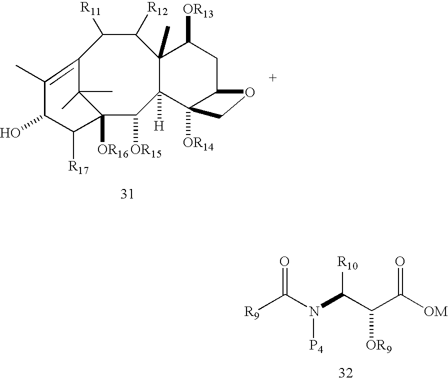
and M is H or an alkali metal selected from the group consisting of Li, Na and K.

41. The process of claim 40, wherein the side chain is compound 32;
P₄ and R₉ together with the nitrogen and oxygen to which they are attached form a cyclic 2,4-dimethoxy benzylidene N,O-acetal or a cyclic 2,6-dimethoxy benzylidene N,O-acetal;
and M is H or is Na or K; and R₁₀ is C₁-C₆ alkyl or phenyl.

42. The process of claim 40, wherein the coupling reagent is selected from the group consisting of acid anhydrides, dicarbonates, chloroformates, acid halides, chlorosulfonates, sulfonic anhydrides, alkyl isocyanates and aryl isocyanates.

43. The process of claim 40, wherein the coupling condition comprises THF or toluene or mixtures thereof, and NMM and DMAP.

44. The process of claim 40, wherein the coupling reagent is selected from the group consisting of benzoic anhydride, phenoxyacetic anhydride, trifluoroacetic anhydride, trimethylacetic anhydride, acetic anhydride, hexanoic anhydride, benzyl chloroformate, trichloroethyl chloroformate, methyl chloroformate, 4-nitrophenyl chloroformate, benzoyl chloride, 2-methoxybenzoyl chloride, 2-chloro-2,2-diphenylbenzoyl chloride, 2,4,6-trichlorobenzoyl chloride, pentafluorobenzoyl chloride, 4-nitro-benzoyl chloride, 2-chloro-benzoyl chloride, phenoxyacetyl chloride, 4-chloromethyl-benzoyl chloride, acetyl chloride, trimethylacetyl chloride, hexanoyl chloride, trimethylacetyl chloride, methane sulfonyl chloride, p-tosyl chlorothionoformate, phenylisocyanate and p-toluene sulfonic anhydride.

45. The process of claim 40, wherein the coupling-reagent is selected from the group consisting of benzoic anhydride, 2,4,6-trichlorobenzoyl chloride and di-t-butyl dicarbonate.

46. The process of claim 40, wherein the coupling product from the coupling reaction is further hydrolyzed to form compound 30 in >95% yield, wherein P₄ and R₉ are hydrogen.

47. A compound selected from the formulae:

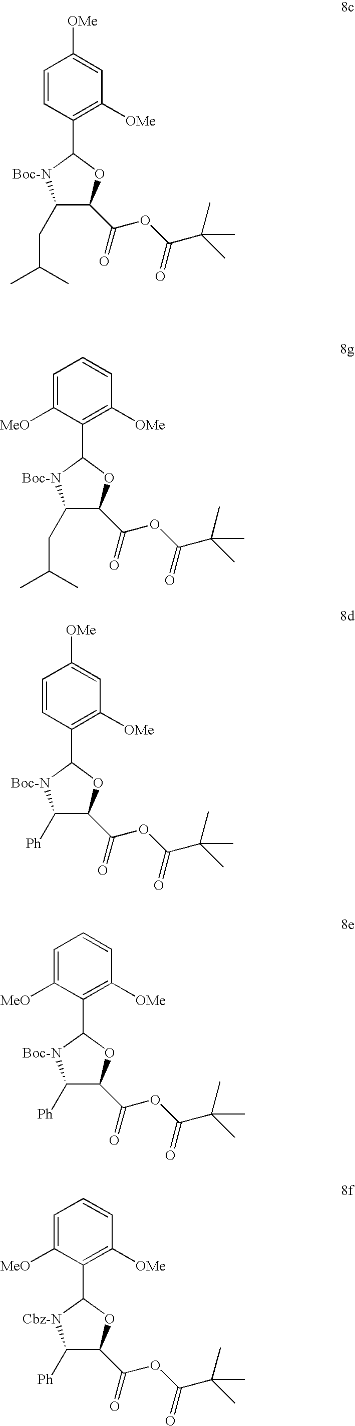

48. A compound selected from the formulae: 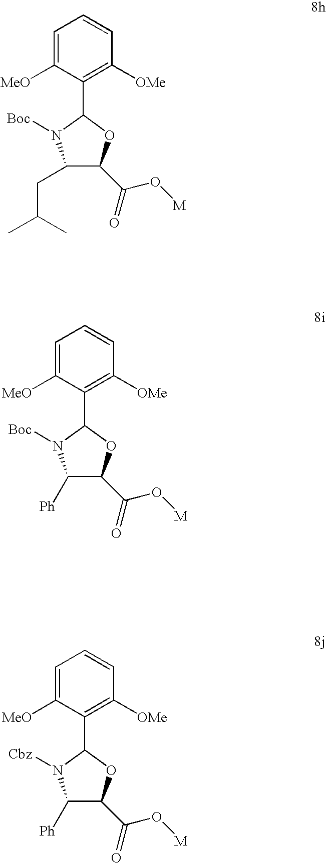
where M is H or a metal selected from the group consisting of Li, Na and K.

49. A compound comprising the formulae: 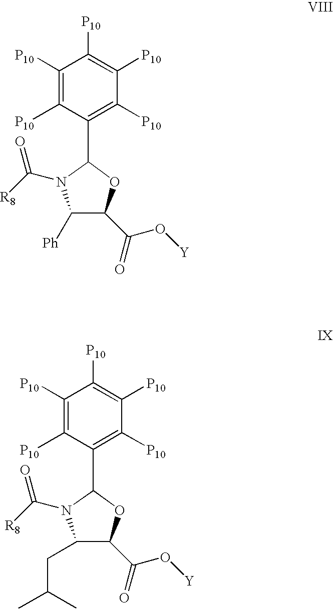
wherein:
R₈ is selected from the group consisting of C₁-C₆ alkoxy, aryloxy, C₁-C₆ alkyl, arylCH₂O—, aryl and heteroaryl;
each P₁₀ is independently H or is an electron donating or electron withdrawing substituent; 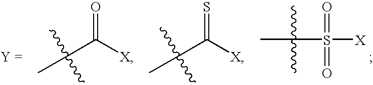
and
X is selected from the group consisting of substituted or unsubstituted C₁-C₁₂ alkyl, C₂-C₁₀ alkenyl, aryl and heteroaryl.

50. A compound comprising the formula: 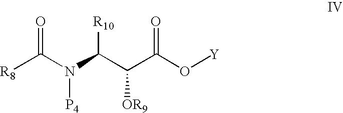
wherein:
R₈ is selected from the group consisting of C₁-C₆ alkoxy, aryloxy, C₁-C₆ alkyl, arylCH₂O—, aryl and heteroaryl;
R₉ is hydrogen or is selected from the group consisting of BOM, Bn, P₃ and a hydroxyl protecting group;
P₄ is H, or P₄ and P₃ together with the nitrogen and oxygen to which P₄ and P₃ are attached form a substituted or unsubstituted cyclic C₁-C₆ alkyl, C₂-C₁₀ alkenyl or aryl acetal, or benzylidene N,O-acetal;
R₁₀ is H or is selected from the group consisting of C₁-C₆ alkyl, C₂-C₁₀ alkenyl, aryl and heteroaryl; 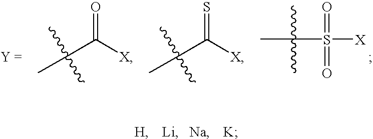
X is selected from the group consisting of substituted or unsubstituted C₁-C₁₂ alkyl, C₂-C₁₀ alkenyl, aryl and heteroaryl; and
X′ is selected from substituted or unsubstituted aryl and heteroaryl;
provided that when Y is H, Li, Na or K and when R₁₀ is isobutyl or phenyl, then P₄ and P₃ together with the nitrogen and oxygen to which P₄ and P₃ are attached are not a cyclic benzylidene N,O-acetal, a cyclic 2,4-dimethoxy benzylidene N,O-acetal, a cyclic 3,4-dimethoxy benzylidene N,O-acetal or a cyclic 4-methoxy benzylidene acetal.
